(19) United States
(12) Patent Application Publication
(10) Pub. No.: US 2022/0403359 A1
(43) Pub. Date: Dec. 22, 2022

(54) VARIANT MALTOPENTAOSE/MALTOHEXAOSE-FORMING ALPHA-AMYLASES

(71) Applicant: DANISCO US INC, Palo Alto, CA (US)

(72) Inventors: MANASI BHATE, SAN FRANCISCO, CA (US); HON KIT CHAN, SAN FRANCISCO, CA (US); JONATHAN LASSILA, SAN FRANCISCO, CA (US); BRIAN JAMES PAUL, WILMINGTON, DE (US); SANDRA W. RAMER, SUNNYVALE, CA (US); PATRICIA TRAN, SAN JOSE, CA (US)

(21) Appl. No.: 17/770,902

(22) PCT Filed: Oct. 20, 2020
(86) PCT No.: PCT/US2020/056428

§ 371 Date: Apr. 21, 2022

Related U.S. Application Data
(60) Provisional application No. 62/925,273, filed on Oct. 24, 2019.

(51) Int. Cl. C12N 9/26; C12N 9/54; C11D 3/386

(52) U.S. Cl. CPC C12N 9/2414; C12Y 302/01001; C12N 9/54; C12Y 304/21062; C11D 3/386

(57) ABSTRACT

Disclosed are compositions and methods relating to maltopentaose/maltohexaose-forming α-amylases. The variant α-amylases are useful, for example, for starch liquefaction and saccharification, for cleaning starchy stains in laundry, dishwashing, and other applications, for textile processing (e.g., desizing), in animal feed for improving digestibility, and for baking and brewing.

### FIELD OF THE INVENTION

Disclosed are compositions and methods relating to variant maltopentaose/maltohexaose-forming α-amylases. The variant α-amylases are useful, for example, for cleaning starchy stains, starch liquefaction and saccharification, textile desizing, baking, and brewing.

### BACKGROUND

Starch consists of a mixture of amylose (15-30% w/w) and amylopectin (70-85% w/w). Amylose consists of linear chains of α-1,4-linked glucose units having a molecular weight (MW) from about 60,000 to about 800,000. Amylopectin is a branched polymer containing α-1,6-branch points every 24-30 glucose units; its MW may be as high as 100 million.

α-amylases hydrolyze starch, glycogen, and related polysaccharides by cleaving internal α-1,4-glucosidic bonds at random. α-amylases, particularly from Bacilli, have been used for a variety of different purposes, including starch liquefaction and saccharification, textile desizing, starch modification in the paper and pulp industry, brewing, baking, production of syrups for the food industry, production of feed-stocks for fermentation processes, and in animal feed to increase digestability. These enzymes can also be used to remove starchy soils and stains during dishwashing and laundry washing.

The products produced by the hydrolysis of starch by α-amylases vary in terms of the number of contiguous glucose molecules. Most commercial α-amylases produce a range of products from glucose (G1) to maltoheptaose (G7). For reasons that are not entirely clear, α-amylases that produce significant amounts of maltopentaose and maltohexaose appear to be especially useful for certain commercial applications, including incorporation into detergent cleaning compositions. Numerous publications have described mutations in maltopentaose/maltohexaose-producing α-amylases and others. Nonetheless, the need continues to exist for ever-more robust and better performing engineered α-amylases molecules.

### SUMMARY

The present compositions and methods relate to variant maltopentaose/maltohexaose-forming amylase polypeptides, and methods of use, thereof. Aspects and embodiments of the present compositions and methods are summarized in the following separately-numbered paragraphs:

1. In one aspect, a recombinant, variant of a parent, non-naturally-occurring α-amylase molecule is provided, comprising a mutation at position 91 and a mutation at an amino acid residue at the base of the α-amylase TIM barrel structure, defined as residues 6, 7, 40, 96, 98, 100, 229, 230, 231, 262, 263, 285, 286, 287, 288, 322, 323, 324, 325, 362, 363 and 364, referring to SEQ ID NO: 1 for numbering, wherein the wild-type amino acid residue present at position 28 of the parent molecule is capable of taking on a positive charge

2. In some embodiments of the variant α-amylase of paragraph 1, the mutation at position 91 is substitution of the naturally-present residue to a positively-charged residue.

3. In some embodiments of the variant α-amylase of paragraphs 1 or 2, the mutation at position 91 is substitution of the naturally-present residue to arginine (i.e., X91R).

4. In some embodiments of the variant α-amylase of any of paragraphs 1-3, the at least one mutation at the base of the α-amylase TIM barrel structure is selected from the group consisting of X40N, X40D, X100F, X100L, X263Y, X288D, X288K, X288Q, X324R, X324N, X324M, X364L and X364M.

5. In some embodiments of the variant α-amylase of any of paragraphs 1-4, the at least one mutation at the base of the α-amylase TIM barrel structure is selected from the group consisting of T40N, T40D, Y100F, Y100L, F263Y, S288D, S288K, S288Q, I324R, I324N, I324M, Y364L and Y364M.

6. In another aspect, a recombinant, variant, non-naturally-occurring α-amylase is provided, comprising an arginine at position 91 and at least one of the following features not present in naturally-occurring α-amylase: N or D at position 40, F or L at position 100, Y at position 263, D, K or Q at position 288, R, N or M at position 324 or L or M at position 364.

7. In some embodiments, the variant α-amylase of any of paragraphs 1-6 further comprises a mutation at a residue in the loop comprising surface-exposed residues 167, 169, 171, 172 and 176, referring to SEQ ID NO: 1 for numbering.

8. In some embodiments of the variant α-amylase of paragraph 7, the at least one mutation in the loop is selected from the group consisting of X167F, X169H, X171Y, X172R, X172N and X176S.

9. In some embodiments of the variant α-amylase of paragraph 8, the at least one mutation in the loop is selected from the group consisting of W167F, Q169H, R171Y, Q172R, Q172N and R176S.

10. In some embodiments, the variant α-amylase of any of paragraphs 1-6 further comprises F at position 167, H at position 169, Y at position 171, R or N at position 172 or S at position 176, referring to SEQ ID NO: 1 for numbering.

11. In another aspect, a recombinant, variant, non-naturally-occurring α-amylase is provided, comprising a mutation at position 172 and a mutation at position 288, referring to SEQ ID NO: 1 for numbering.

12. In another aspect, a recombinant, variant, non-naturally-occurring α-amylase is provided, comprising arginine or asparagine at position 172 and aspartic acid at position 288, referring to SEQ ID NO: 1 for numbering.

13. In some embodiments, the variant α-amylase of any of paragraphs 1-12 further comprises a mutation at position 116 and/or 281, referring to SEQ ID NO: 1 for numbering.

14. In some embodiments, the variant α-amylase of any of paragraphs 1-12 further comprises arginine at position 116 or serine at position 281, referring to SEQ ID NO: 1 for numbering.

15. In some embodiments, the variant α-amylase of any of any of paragraphs 1-14 further comprises a mutation at position 190 and/or 244, referring to SEQ ID NO: 1 for numbering.

16. In some embodiments, the variant α-amylase of any of any of paragraphs 1-14 has proline at position 190 is and/or alanine, glutamic acid or glutamine at position 244, referring to SEQ ID NO: 1 for numbering.

17. In some embodiments, the variant α-amylase of any of paragraphs 1-16 further comprises deletion of at least two residues equivalent to R181, G182, T183, and G184, using SEQ ID NO: 1.

18. In some embodiments, the variant α-amylase of any of paragraphs 1-16 further comprises pairwise deletions of residues equivalent to R181 and G182 or to residues T183 and G184.

19. In another aspect, a recombinant, variant, non-naturally-occurring α-amylase is provided, comprising:

(i) substitutions selected from the group consisting of:
(a) X40N-X91R-X169H-X183M-X281N,
(b) X172R-X190P-X288D,
(c) X172R-X244E-X288D-X474R,
(d) X91R-X172R-X190P-X324M,
(e) X40N-X91R-X190P-X263Y,
(f) X40N-X91R-X244E-X364L,
(g) X91R-X172R-X190P-X324R,
(h) X91R-X116R-X172R-X244E-X281S-X288D,
(i) X40N-X91R-X100E-X116R-X172N-X244Q-X281S,
(j) X40N-X91R-X172R-X244Q-X263Y-X281S,
(k) X91R-X172R-X190P-X324N,
(l) X40D-X91R-X172R-X190P-X281S-X324R, and
(m) X364L; and

(ii) pairwise deletions of residues selected from the group consisting of residues equavalent to:
181 and 182, and
183 and 184,

using SEQ ID NO: 1 for numbering.

20. In some embodiments, the variant, α-amylase of paragraph 19 comprises:

(i) substitutions selected from the group consisting of:
(a) T40N-S91R-Q169H-T183M-H281N,
(b) Q172R-E190P-S288D,
(c) Q172R-S244E-S288D-S474R,
(d) S91R-Q172R-E190P-I324M,
(e) T40N-S91R-E190P-F263Y,
(f) T40N-S91R-S244E-Y364L,
(g) S91R-Q172R-E190P-I324R,
(h) S91R-W116R-Q172R-S244E-H281S-S288D,
(i) T40N-S91R-Y100E-W116R-Q172N-S244Q-H281S,
(j) T40N-S91R-Q172R-S244Q-F263Y-H281S,
(k) S91R-Q172R-E190P-I324N,
(1) T40D-S91R-Q172R-E190P-H281S-I324R, and
(m) Y364L; and

(ii) pairwise deletions of residues selected from the group consisting of:
R181 and G182, and
T183 and G184,

using SEQ ID NO: 1 for numbering.

21. In another aspect, a recombinant, variant, non-naturally-occurring α-amylase is provided, comprising three or more of the following features: (a) D or N at position 40 and/or R at position 91 and (b) F at position 100, Y at position 263, D at position 288, M, N or R at position 324 and/or L at position 364, optionally in combination with (c) H at position 169, M at position 183M, N or S at position 281, N or R at position 172, P at position 190, E, Q or Rat position 244, R at position 474 and/or R at postion 116, (d) optionally in combination with (e) pairwise deletions at positions 181 and 182 or 183 and 184, in all cases using SEQ ID NO: 1 for numbering.

22. In some embodiments, the variant α-amylase of any of paragraphs 1-21 has at least 70%, at least 80%, at least 90% or at least 95% amino acid sequence identity to the amino acid sequence of SEQ ID NO: 1, SEQ ID NO: 2, SEQ ID NO: 3 or SEQ ID NO: 4.

23. In another aspect, a detergent composition comprising the variant α-amylase of any of paragraphs 1-22 is provided.

24. In some embodiments, the detergent composition of paragraph 23 further comprises a variant subtilisin protease from Bacillus gibsonii having the amino acid substitutions X39E, X99R, X126A, X127E and X128G, and further comprising one or more additional substitutions selected from the group consisting of N74D-M211L-N253P, R179Q-M211L-N253P, N74D-N253P, N85R-G160Q-R179Q-M211L-N212S-N253P, R179Q-N253P, G160Q-R179Q-M211L-N212S-N253P, R179Q-M211L, G160Q-R179Q-M211L-N253P, G160Q-R179Q-N212S-N253P, N74D-M211L, M211L-N242D, G160Q-R179Q-M211L-N212S, N74D-R179Q-M211L-N253P, G160Q-R179Q-M211L, G160Q-R179Q-N253P, N74D-Q200L-M211L, N74D-G160Q-N212S-N253P, N74D-G160Q-M211L-N253P, G160Q-R179Q, G160Q-R179Q-N212S, N74D-G160Q-N253P, N74D-G160Q-R179Q-M211L-N212S-N253P, N74D-N085R-G160Q-R179Q-M211L, N74D-G160Q-M211L-N212S-N253P, N74D-N085R-N116R-Q200L-Q256E, N74D-G160Q-R179Q-N212S-N253P, N74D-G160Q-M211L-N212S, N74D-G160Q, N74D-G160Q-R179Q-M211L-N253P, N74D-R179Q-M211L, N74D-G160Q-N212S, N74D-G160Q-M211L, N74D-G160Q-R179Q-N253P, N74D, N74D-G160Q-R179Q-M211L-N212S, N74D-N085R-M211L-N212S, N74D-G160Q-R179Q-N212S, N74D-G160Q-R179Q-M211L, N74D-M211L-Q256E, N74D-G160Q-R179Q, R179Q-M211L-N212S-N253P, R179Q-M211L-N212S, N74D-N085R-R179Q-M211L-N212S, N74D-M211L-N212S, N74D-R179Q-M211L-N212S, N74D-M211L-N242D, N74D-Q200L-M211L-Q256E, N74D-Q200L-M211L-N242D-Q256E, N74D-Q200L, N74D-M211N-N212Q, N74D-M211N-N212Q-Q256E, N74D-M211N-Q256E, N74D-M211Q, N74D-M211Q-N212Q, N74D-M211Q-N212Q-Q256E, N74D-M211Q-Q256E, N74D-N198A-M211Q, N74D-N198A-M211Q-N212Q, N74D-N198A-M211Q-Q256E, N74D-N198G-M211Q, N74D-N198G-M211Q-N212Q, N74D-N198G-M211Q-Q256E, N74D-N198K-M211Q-N212Q, N74D-N198L-M211Q-N212Q, N74D-N198Q-M211Q-N212Q, N74D-N198R-M211Q-N212Q, N74D-N198T-M211Q-N212Q, N74D-N198V-M211Q-N212Q, N74D-N212Q-Q256E, N74D-Q256E, N74D-R207Q, N74D-R207Q-M211N, N74D-R207Q-M211N-N212Q, N74D-R207Q-M211N-N212Q-Q256E, N74D-R207Q-M211N-Q256E, N74D-R207Q-M211Q, N74D-R207Q-M211Q-N212Q, N74D-R207Q-M211Q-N212Q-Q256E, N74D-R207Q-N212Q, N74D-R207Q-N212Q-Q256E, N74D-R207Q-Q256E, N74D-N198S-M211Q and N74D-N198L-M211Q, referring to SEQ ID NO: 5 for numbering and having at least 90% amino acid sequence identity to SEQ ID NO: 6.

25. In another aspect, a method for converting starch to oligosaccharides is provided, comprising contacting starch with effective amount of the variant α-amylase of any of paragraphs 1-22.

26. In another aspect, a method for removing a starchy stain or soil from a surface is provided, comprising contacting the surface with an effective amount of the variant α-amylase of any of paragraphs 1-22, and allowing the polypeptide to hydrolyze starch components present in the starchy stain to produce smaller starch-derived molecules that dissolve in the aqueous composition, thereby removing the starchy stain from the surface.

27. In another aspect, a nucleic acid encoding the variant α-amylase of any of paragraphs 1-22 is provided.

28. In another aspect, a host cell comprising the nucleic acid of paragraph 27 is provided.

These and other aspects and embodiments of the present compositions and methods will be apparent from the following description and appended Examples.

### BRIEF DESCRIPTION OF THE DRAWINGS

FIG. 1 shows a Clustal W amino acid sequence alignment of AA2560, AA707, AA560 and AAI10.

FIG. 2 shows a view of AA2560 amylase through the central β-barrel. Residues at the bottom of the barrel are shown with the the α-carbon positions rendered in spheres. The amino acid numbering is given for these positions.

FIG. 3 shows the AA2560 amylase in a side view of the central β-barrel, oriented with the helix at amino acids 82-94 in front. Residues at the bottom of the barrel are shown with the the α-carbon positions rendered in spheres and the position number indicated.

FIG. 4 is a cutaway view showing two helices within the structural model of AA2560. Positions 28 and 91 are shown in stick representation. The left image shows wild-type Arg28 and Ser91 in stick representation. The right image shown the close positioning of two Arg residues in the S91R variant in stick representation.

FIG. 5 is a Table showing the performance of AA2560, AA560, AA707 and AAI10 comninatorial variants in a cleaning assay.

### DETAILED DESCRIPTION

Described are compositions and methods relating to variant maltopentaose/maltohexaose-forming amylase enzymes. The variants were discovered by experimental approaches as detailed in the appended Examples. Exemplary applications for the variant amylase enzymes are for cleaning starchy stains in dishwashing, laundry and other applications, for starch liquefaction and saccharification, for textile processing (e.g., desizing), in animal feed for improving digestibility, and and for baking and brewing. These and other aspects of the compositions and methods are described in detail, below.

Prior to describing the various aspects and embodiments of the present compositions and methods, the following definitions and abbreviations are described.

### 1. Definitions and Abbreviations

In accordance with this detailed description, the following abbreviations and definitions apply. Note that the singular forms “a,” “an,” and “the” include plural referents unless the context clearly dictates otherwise. Thus, for example, reference to “an enzyme” includes a plurality of such enzymes, and reference to “the dosage” includes reference to one or more dosages and equivalents thereof known to those skilled in the art, and so forth.

The present document is organized into a number of sections for ease of reading; however, the reader will appreciate that statements made in one section may apply to other sections. In this manner, the headings used for different sections of the disclosure should not be construed as limiting.

Unless defined otherwise, all technical and scientific terms used herein have the same meaning as commonly understood by one of ordinary skill in the art. The following terms are defined, below, for clarity.

### 1.1. Abbreviations and Acronyms

The following abbreviations/acronyms have the following meanings unless otherwise specified:

° C. degrees Centigrade

ADW automatic dishwashing

dH₂O or DI deionized water

dIH₂O deionized water, Milli-Q filtration

DNA deoxyribonucleic acid

EC Enzyme Commission

g or gm grams

GA glucoamylase

H₂O water

HDD heavy duty powder detergent

HDL high density liquid detergent

hr(s) hour/hours

HSG high suds granular detergent

kDa kiloDalton

kg kilograms

M molar

mg milligrams

min(s) minute/minutes

mL and ml milliliters

mm millimeters

mM millimolar

MW molecular weight

MWU modified Wohlgemuth unit; 1.6×10⁻⁵ mg/MWU=unit of activity

PI performance index

ppm parts per million, e.g., μg protein per gram dry solid

sec seconds

sp. species

U units

v/v volume/volume

w/v weight/volume

w/w weight/weight

wt % weight percent

micrograms

μL and μl microliters

μm a micrometer

μM micromolar

### 1.2. Definitions

The terms “α-amylase” or “amylolytic enzyme” or generally amylase refer to an enzyme that is, among other things, capable of catalyzing the degradation of starch. α-Amylases are hydrolases that cleave the α-D-(1→4) O-glycosidic linkages in starch. Generally, α-amylases (EC 3.2.1.1; α-D-(1→4)-glucan glucanohydrolase) are defined as endo-acting enzymes cleaving α-D-(1→4) O-glycosidic linkages within the starch molecule in a random fashion yielding polysaccharides containing three or more (1-4)-α-linked D-glucose units. In contrast, the exo-acting amylolytic enzymes, such as β-amylases (EC 3.2.1.2; α-D-(1→4)-glucan maltohydrolase) and some product-specific α-amylases like maltogenic α-amylase (EC 3.2.1.133) cleave the polysaccharide molecule from the non-reducing end of the substrate. β-amylases, α-glucosidases (EC 3.2.1.20; α-D-glucoside glucohydrolase), glucoamylase (EC 3.2.1.3; α-D-(1→4)-glucan glucohydrolase), and product-specific amylases like the maltotetraosidases (EC 3.2.1.60) and the maltohexaosidases (EC 3.2.1.98) can produce malto-oligosaccharides of a specific length or enriched syrups of specific maltooligosaccharides. Some bacterial α-amylases predominantly produce maltotetraose (G4), maltopentaose (G5) or maltohexaose (G6) from starch and related α-1,4-glucans, while most α-amylases further convert them to glucose and or maltose as final products. G6 amylases such as AA560 amylase derived from Bacillus sp. DSM 12649 (i.e., the parent of STAINZYME™) and Bacillus sp. 707 amylase, which are also called maltohexaose-forming α-amylases (EC 3.2.1.98), are technically exo acting, but have similar structures compared to α-amylases, and in some cases appear to respond to the some of the same beneficial mutations.

“Enzyme units” herein refer to the amount of product formed per time under the specified conditions of the assay. For example, a “glucoamylase activity unit” (GAU) is defined as the amount of enzyme that produces 1 g of glucose per hour from soluble starch substrate (4% DS) at 60° C., pH 4.2. A “soluble starch unit” (SSU) is the amount of enzyme that produces 1 mg of glucose per minute from soluble starch substrate (4% DS) at pH 4.5, 50° C. DS refers to “dry solids.”

The term “starch” refers to any material comprised of the complex polysaccharide carbohydrates of plants, comprised of amylose and amylopectin with the formula (C₆H₁₀O₅)ₓ, wherein X can be any integer. The term includes plant-based materials such as grains, cereal, grasses, tubers and roots, and more specifically materials obtained from wheat, barley, corn, rye, rice, sorghum, brans, cassava, millet, milo, potato, sweet potato, and tapioca. The term “starch” includes granular starch. The term “granular starch” refers to raw, i.e., uncooked starch, e.g., starch that has not been subject to gelatinization.

As used herein, the term “liquefaction” or “liquefy” means a process by which starch is converted to less viscous and shorter chain dextrins.

The terms, “wild-type,” “parental,” or “reference,” with respect to a polypeptide, refer to a naturally-occurring polypeptide that does not include a man-made substitution, insertion, or deletion at one or more amino acid positions. Similarly, the terms “wild-type,” “parental,” or “reference,” with respect to a polynucleotide, refer to a naturally-occurring polynucleotide that does not include a man-made nucleoside change. However, note that a polynucleotide encoding a wild-type, parental, or reference polypeptide is not limited to a naturally-occurring polynucleotide, and encompasses any polynucleotide encoding the wild-type, parental, or reference polypeptide.

Reference to the wild-type polypeptide is understood to include the mature form of the polypeptide. A “mature” polypeptide or variant, thereof, is one in which a signal sequence is absent, for example, cleaved from an immature form of the polypeptide during or following expression of the polypeptide.

The term “variant,” with respect to a polypeptide, refers to a polypeptide that differs from a specified wild-type, parental, or reference polypeptide in that it includes one or more naturally-occurring or man-made substitutions, insertions, or deletions of an amino acid. Similarly, the term “variant,” with respect to a polynucleotide, refers to a polynucleotide that differs in nucleotide sequence from a specified wild-type, parental, or reference polynucleotide. The identity of the wild-type, parental, or reference polypeptide or polynucleotide will be apparent from context.

In the case of the present α-amylases, “activity” refers to α-amylase activity, which can be measured as described, herein.

The term “performance benefit” refers to an improvement in a desirable property of a molecule. Exemplary performance benefits include, but are not limited to, increased hydrolysis of a starch substrate, increased grain, cereal or other starch substrate liquifaction performance, increased cleaning performance, increased thermal stability, increased detergent stability, increased storage stability, increased solubility, an altered pH profile, decreased calcium dependence, increased specific activity, modified substrate specificity, modified substrate binding, modified pH-dependent activity, modified pH-dependent stability, increased oxidative stability, and increased expression. In some cases, the performance benefit is realized at a relatively low temperature. In some cases, the performance benefit is realized at relatively high temperature.

The terms “protease” and “proteinase” refer to an enzyme protein that has the ability to perform “proteolysis” or “proteolytic cleavage” which refers to hydrolysis of peptide bonds that link amino acids together in a peptide or polypeptide chain forming the protein. This activity of a protease as a protein-digesting enzyme is referred to as “proteolytic activity.”

The terms “serine protease” refers to enzymes that cleave peptide bonds in proteins, in which enzymes serine serves as the nucleophilic amino acid at the enzyme active site. Serine proteases fall into two broad categories based on their structure: chymotrypsin-like (trypsin-like) or subtilisin-like. Most commonly used in laundry and dishwashing detergents are serine protease, particularly subtlisins.

“Combinatorial variants” are variants comprising two or more mutations, e.g., 2, 3, 4, 5, 6, 7, 8, 9, 10, or more, substitutions, deletions, and/or insertions.

The term “recombinant,” when used in reference to a subject cell, nucleic acid, protein or vector, indicates that the subject has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding an amylase is a recombinant vector.

The terms “recovered,” “isolated,” and “separated,” refer to a compound, protein (polypeptides), cell, nucleic acid, amino acid, or other specified material or component that is removed from at least one other material or component with which it is naturally associated as found in nature. An “isolated” polypeptides, thereof, includes, but is not limited to, a culture broth containing secreted polypeptide expressed in a heterologous host cell.

The term “purified” refers to material (e.g., an isolated polypeptide or polynucleotide) that is in a relatively pure state, e.g., at least about 90% pure, at least about 95% pure, at least about 98% pure, or even at least about 99% pure.

The term “enriched” refers to material (e.g., an isolated polypeptide or polynucleotide) that is in about 50% pure, at least about 60% pure, at least about 70% pure, or even at least about 70% pure.

The terms “thermostable” and “thermostability,” with reference to an enzyme, refer to the ability of the enzyme to retain activity after exposure to an elevated temperature. The thermostability of an enzyme, such as an amylase enzyme, is measured by its half-life (t½) given in minutes, hours, or days, during which half the enzyme activity is lost under defined conditions. The half-life may be calculated by measuring residual α-amylase activity following exposure to (i.e., challenge by) an elevated temperature.

A “pH range,” with reference to an enzyme, refers to the range of pH values under which the enzyme exhibits catalytic activity.

The terms “pH stable” and “pH stability,” with reference to an enzyme, relate to the ability of the enzyme to retain activity over a wide range of pH values for a predetermined period of time (e.g., 15 min., 30 min., 1 hour).

The term “amino acid sequence” is synonymous with the terms “polypeptide,” “protein,” and “peptide,” and are used interchangeably. Where such amino acid sequences exhibit activity, they may be referred to as an “enzyme.” The conventional one-letter or three-letter codes for amino acid residues are used, with amino acid sequences being presented in the standard amino-to-carboxy terminal orientation (i.e., N→C).

The term “nucleic acid” encompasses DNA, RNA, heteroduplexes, and synthetic molecules capable of encoding a polypeptide. Nucleic acids may be single stranded or double stranded, and may contain chemical modifications. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

A “synthetic” molecule is produced by in vitro chemical or enzymatic synthesis rather than by an organism.

The term “introduced” in the context of inserting a nucleic acid sequence into a cell, means “transfection”, “transformation” or “transduction,” as known in the art.

A “host strain” or “host cell” is an organism into which an expression vector, phage, virus, or other DNA construct, including a polynucleotide encoding a polypeptide of interest (e.g., an amylase) has been introduced. Exemplary host strains are microorganism cells (e.g., bacteria, filamentous fungi, and yeast) capable of expressing the polypeptide of interest and/or fermenting saccharides. The term “host cell” includes protoplasts created from cells.

The term “heterologous” with reference to a polynucleotide or protein refers to a polynucleotide or protein that does not naturally occur in a host cell.

The term “endogenous” with reference to a polynucleotide or protein refers to a polynucleotide or protein that occurs naturally in the host cell.

The term “expression” refers to the process by which a polypeptide is produced based on a nucleic acid sequence. The process includes both transcription and translation.

A “signal sequence” is a sequence of amino acids attached to the N-terminal portion of a protein, which facilitates the secretion of the protein outside the cell. The mature form of an extracellular protein lacks the signal sequence, which is cleaved off during the secretion process.

“Biologically active” refer to a sequence having a specified biological activity, such an enzymatic activity.

The term “specific activity” refers to the number of moles of substrate that can be converted to product by an enzyme or enzyme preparation per unit time under specific conditions. Specific activity is generally expressed as units (U)/mg of protein.

As used herein, “water hardness” is a measure of the minerals (e.g., calcium and magnesium) present in water.

“A cultured cell material comprising an amylase” or similar language, refers to a cell lysate or supernatant (including media) that includes an amylase as a component. The cell material may be from a heterologous host that is grown in culture for the purpose of producing the amylase.

“Percent sequence identity” means that a particular sequence has at least a certain percentage of amino acid residues identical to those in a specified reference sequence, when aligned using the CLUSTAL W algorithm with default parameters. See Thompson et al. (1994) Nucleic Acids Res. 22:4673-4680. Default parameters for the CLUSTAL W algorithm are:
Gap opening penalty: 10.0
Gap extension penalty: 0.05
Protein weight matrix: BLOSUM series
DNA weight matrix: IUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty OFF

Deletions are counted as non-identical residues, compared to a reference sequence.

The term “dry solids content” (ds) refers to the total solids of a slurry in a dry weight percent basis. The term “slurry” refers to an aqueous mixture containing insoluble solids.

The phrase “simultaneous saccharification and fermentation (SSF)” refers to a process in the production of biochemicals in which a microbial organism, such as an ethanologenic microorganism, and at least one enzyme, such as an amylase, are present during the same process step. SSF includes the contemporaneous hydrolysis of starch substrates (granular, liquefied, or solubilized) to saccharides, including glucose, and the fermentation of the saccharides into alcohol or other biochemical or biomaterial in the same reactor vessel.

An “ethanologenic microorganism” refers to a microorganism with the ability to convert a sugar or oligosaccharide to ethanol.

The term “fermented beverage” refers to any beverage produced by a method comprising a fermentation process, such as a microbial fermentation, e.g., a bacterial and/or fungal fermentation.

The term “malt” refers to any malted cereal grain, such as malted barley or wheat.

The term “mash” refers to an aqueous slurry of any starch and/or sugar containing plant material, such as grist, e.g., comprising crushed barley malt, crushed barley, and/or other adjunct or a combination thereof, mixed with water later to be separated into wort and spent grains.

The term “wort” refers to the unfermented liquor run-off following extracting the grist during mashing.

The term “about” refers to ±15% to the referenced value.

### 2. Maltopentaose/Maltohexaose-Forming α-Amylase Variants

Described are combinatorial variants of maltopentaose/maltohexaose-forming α-amylases that show a high degree of performance in automatic dishwashing (ADW) applications The variants are most closely related to an α-amylase from a Bacillus sp., herein, refered to as AA2560, and previously identified as BspAmy24 (SEQ ID NO: 1) in WO 2018/184004. The mature amino acid sequence of AA2560 α-amylase is shown, below, as SEQ ID NO: 1:

HHNGTNGTMM QYFEWHLPND GQHWNRLRND AANLKNLGIT
AVWIPPAWKG TSQNDVGYGA YDLYDLGEFN QKGTIRTKYG
TRSQLQSAIA SLQNNGIQVY GDVVMNHKGG ADGTEWVQAV
EVNPSNRNQE VTGEYTIEAW TKFDFPGRGN THSSFKWRWY
HFDGTDWDQS RQLNNRIYKF RGTGKAWDWE VDTENGNYDY
LMYADVDMDH PEVINELRRW GVWYTNTLNL DGFRIDAVKH
IKYSFTRDWL NHVRSTTGKN NMFAVAEFWK NDLGAIENYL
HKTNWNHSVF DVPLHYNLYN ASKSGGNYDM RQILNGTVVS
KHPIHAVTFV DNHDSQPAEA LESFVEAWFK PLAYALILTR
EQGYPSVFYG DYYGIPTHGV AAMKGKIDPI LEARQKYAYG
TQHDYLDHHN IIGWTREGNS AHPNSGLATI MSDGPGGSKW
MYVGRHKAGQ VWRDITGNRT GTVTINADGW GNFSVNGGSV
SIWVNK

A closely related maltopentaose/maltohexaose-forming α-amylase is from Bacillus sp. 707, herein, refered to as “AA707.” The mature amino acid sequence of AA707 α-is shown, below, as SEQ ID NO: 2:

HHNGTNGTMM QYFEWYLPND GNHWNRLNSD ASNLKSKGIT
AVWIPPAWKG ASQNDVGYGA YDLYDLGEFN QKGTVRTKYG
TRSQLQAAVT SLKNNGIQVY GDVVMNHKGG ADATEMVRAV
EVNPNNRNQE VTGEYTIEAW TRFDFPGRGN THSSFKWRWY
HFDGVDWDQS RRLNNRIYKF RGHGKAWDWE VDTENGNYDY
LMYADIDMDH PEVVNELRNW GVWYTNTLGL DGFRIDAVKH
IKYSFTRDWI NHVRSATGKN MFAVAEFWKN DLGAIENYLQ
KTNWNHSVFD VPLHYNLYNA SKSGGNYDMR NIFNGTVVQR
HPSHAVTFVD NHDSQPEEAL ESFVEEWFKP LAYALTLTRE
QGYPSVFYGD YYGIPTHGVP AMRSKIDPIL EARQKYAYGK
QNDYLDHHNI IGWTREGNTA HPNSGLATIM SDGAGGSKWM
FVGRNKAGQV WSDITGNRTG TVTINADGWG NFSVNGGSVS
IWVNK

Another closely related maltopentaose/maltohexaose-forming α-amylase is from a Bacillus sp. referred to as AA560. The mature amino acid sequence of AA560 is shown, below, as SEQ ID NO: 3:

HHNGTNGTMM QYFEWYLPND GNHWNRLRSD ASNLKDKGIS
AVWIPPAWKG ASQNDVGYGA YDLYDLGEFN QKGTIRTKYG
TRNQLQAAVN ALKSNGIQVY GDVVMNHKGG ADATEMVRAV
EVNPNNRNQE VSGEYTIEAW TKFDFPGRGN THSNFKWRWY
HFDGVDWDQS RKLNNRIYKF RGDGKGWDWE VDTENGNYDY
LMYADIDMDH PEVVNELRNW GVWYTNTLGL DGFRIDAVKH
IKYSFTRDWI NHVRSATGKN MFAVAEFWKN DLGAIENYLN
KTNWNHSVFD VPLHYNLYNA SKSGGNYDMR QIFNGTVVQR
HPMHAVTFVD NHDSQPEEAL ESFVEEWFKP LAYALTLTRE
QGYPSVFYGD YYGIPTHGVP AMKSKIDPIL EARQKYAYGR
QNDYLDHHNI IGWTREGNTA HPNSGLATIM SDGAGGNKWM
FVGRNKAGQV WTDITGNRAG TVTINADGWG NFSVNGGSVS
IWVNK

Based on amino acid sequence identity, another postulated maltopentaose/maltohexaose-forming α-amylase is from another Bacillus sp., and is herein referred to as AAI10. The mature amino acid sequence of AAI10 α-amylase is shown, below, as SEQ ID NO: 4:

HHDGTNGTIM QYFEWNVPND GQHWNRLHNN AQNLKNAGIT
AIWIPPAWKG TSQNDVGYGA YDLYDLGEFN QKGTVRTKYG
TKAELERAIR SLKANGIQVY GDVVMNHKGG ADFTERVQAV
EVNPQNRNQE VSGTYQIEAW TGFNFPGRGN QHSSFKWRWY
HFDGTDWDQS RQLANRIYKF RGDGKAWDWE VDTENGNYDY
LMYADVDMDH PEVINELNRW GVWYANTLNL DGFRLDAVKH
IKFSFMRDWL GHVRGQTGKN LFAVAEYWKN DLGALENYLS
KTNWTMSAFD VPLHYNLYQA SNSSGNYDMR NLLNGTLVQR
HPSHAVTFVD NHDTQPGEAL ESFVQGWFKP LAYATILTRE
QGYPQVFYGD YYGIPSDGVP SYRQQIDPLL KARQQYAYGR
QHDYFDHWDV IGWTREGNAS HPNSGLATIM SDGPGGSKWM
YVGRQKAGEV WHDMTGNRSG TVTINQDGWG HFFVNGGSVS
VWVKR

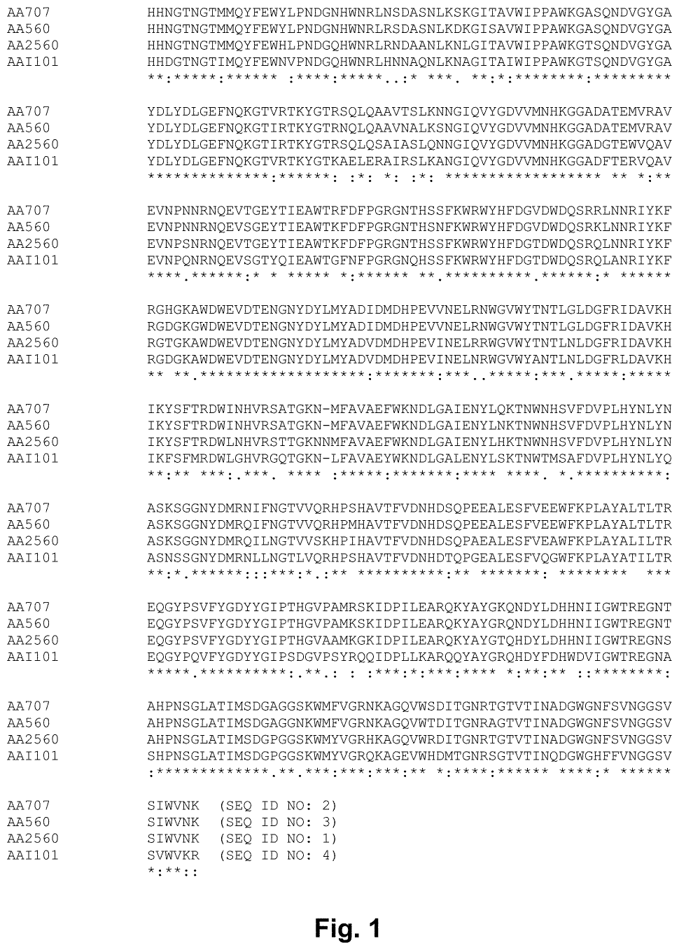

An alignment of these four α-amylases is shown in FIG. 1. Amino acid sequence identity is summarized in Table 1. AA707, AA560 and AAI10 all have greater than 80% amino acid to AA2560.

TABLE 1
Amino acid sequence identity of α-amylase

| | AA2560 | AA707 | AA560 | AAI10 |
|---|---|---|---|---|
| AA2560 | — | 90.3 | 89.5 | 81.7 |
| AA707 | 90.3 | — | 95.5 | 79.8 |
| AA560 | 89.5 | 95.5 | — | 78.6 |
| AAI10 | 81.7 | 79.8 | 78.6 | — |

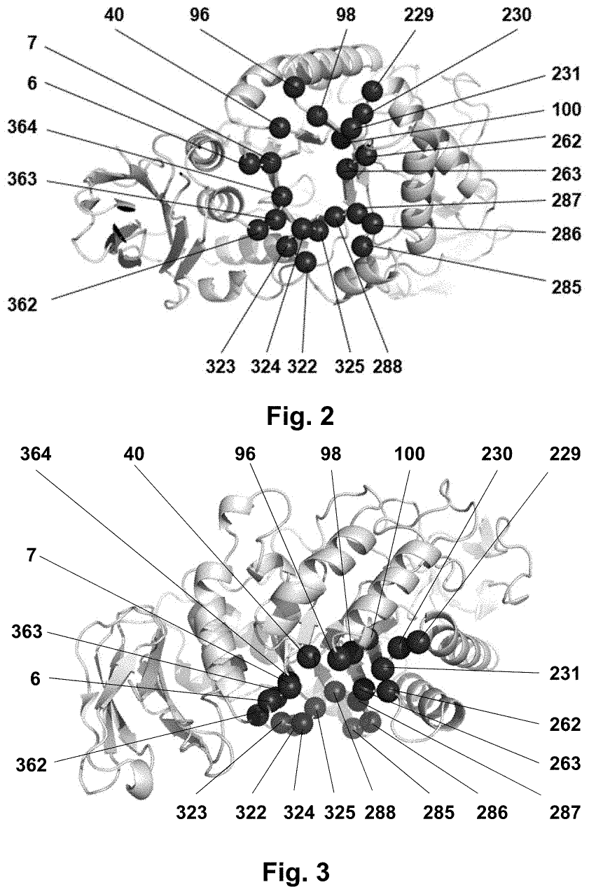

One feature of the present variants is mutation at position 91 and/or at least one mutation at the bottom of the α-amylase TIM barrel structure. The barrel bottom residues have solvent accessible surface area greater than zero and lie in or adjacent to the core β-barrel structure, at the side of the barrel opposite of the active site, and at the side containing the N-terminal ends of each strand. Solvent accessible surface area was calculated using MOE 2018.01 (Chemical Computing Group, Montreal), using default parameters, and based on a homology model of AA2560 constructed with MOE 2018.01 using default parameters and the 1BLI structure from the pdb. Relevant residues are at positions 6, 7, 40, 96, 98, 100, 229, 230, 231, 262, 263, 285, 286, 287, 288, 322, 323, 324, 325, 362, 363 and 364, referring to SEQ ID NO: 1 for numbering. The structural significance of these barrel bottom residues can be appreciated with the help of the images in FIGS. 2 and 3. In all cases, the residues line the base of the TIM barrel structure, which represents a primary architechtural feature of α-amylases and many other enzymes. An exemplary mutation at residue 91 is substitution from a polar residue to a charged residue, particlarly a positively-charged residue, such as arginine (i.e., X91R), which in the case of AA2560 is the specific substitution S91R.

A notable exception, Amy707 differs from AA2560, AAI10, and AA560 (as well as amylases from a Cytophaga sp. (Jeang, C-L. et al. (2002) Applied and Environmental Microbiolgy, 68:3651-54; Genbank Accession number AAA22231), Bacillus sp. TS-23 (Lin, L-L. et al. (1997) J Appl Microbiol, 82:325-34; Genbank Accession number AAA63900) and others), in that it does not have an amino acid residue in position 28 that is capable of taking on a positive charge. AA2560, AAI10, and AA560 have Arg or His sidechains in position 28, whereas Amy707 has Asn, which cannot adopt a positive charge (Table 2), referring to SEQ ID NO: 1 for numbering.

TABLE 2
Amino acid side chains in position 28 of four amylases

| Amylase | Wild-type residue at position 28 |
|---|---|
| AA707 | Asn |
| AA2560 | Arg |
| AAI10 | His |
| AA560 | Arg |

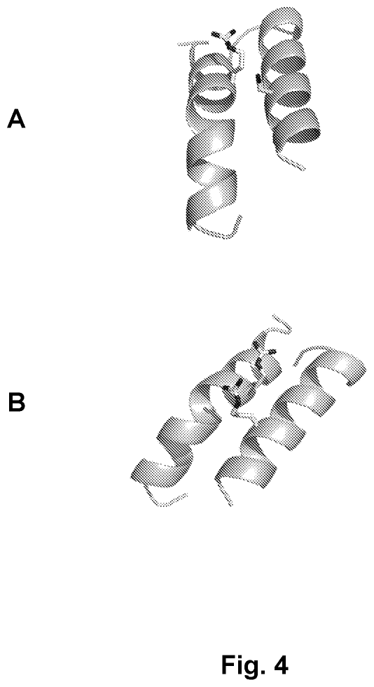

Accordingly, in AA2560, AA560, AAI10, and many other amylases, mutation of Ser91 to Arg would result in very close positioning of two positively charged amino acids, as is apparent from looking at the model of AA2560 shown in FIG. 4. Without being limited to a theory, it is postulated that the close positioning of these residues could favorably impact activity in many amylases such as AA2560 by repositioning the helices at positions 22-37 and 82-95 as a result of the charge-charge repulsion. In contrast, the Asn28 in wild-type Amy707 may be able to hydrogen bond with an Arg in position 91, perhaps resulting a tighter interaction that may be less favorable for activity in Amy707. As above and below, SEQ ID NO: 1 is used for for numbering.

Exemplary mutations in the barrel bottom residues are substitutions, including but not limited to X40N, X40D X100F, X100L, X263Y, X288D, X288K, X288Q, X324R, X324N, X324M, X364L and X364M, where “X” is the previously-existing amino acid residue in the wild-type paental α-amylase. Specific mutations with refernce to AA2560 are T40N, T40D Y100F, Y100L, F263Y, S288D, S288K, S288Q, I324R, I324N, I324M, Y364L and Y364M.

Differently described, the variants have one, two three or more features including N or D at position 40, F or L at position 100, Y at position 263, D, K or Q at position 288, R, N or M at position 324 or L or M at position 364.

While the mutation at position 91 and the mutation at the barrel bottom result in superior perfomance advantages in combination, each mutation alone appears to produce a benefit, and some of the present variants have a mutations at only one position/structure.

The variants may additionally feature mutations in the loop that includes surface-exposed residues 167, 169, 171, 172 and 176, referring to SEQ ID NO: 1 for numbering. Exemplary mutations include but are not limited to the substitutions, X167F, X169H, X171Y, X172R, X172N and X176S and specifically, W167F, Q169H, R171Y, Q172R, Q172N and R176S. Differently described, the variants feature substitutions including F at position 167, H at position 169, Y at position 171, R or N at position 172 and/or S at position 176, referring to SEQ ID NO: 1 for numbering.

The variants may additionally feature mutations at positions 116 and 281, which are believed to affect solubility. Exemplary mutations at these positions are the substitutions X116R and X281S, specifically the substitutions W116R and H281S.

The variants may additionally feature stabilizing mutations at positions 190 and/or 244, referring to SEQ ID NO: 1 for numbering. Such mutations have been well categorized, and are included in current, commercially-available α-amylases used for cleaning, grain processing, and textiles processing. Exemplary mutations in these resudues are the substitutions X190P and X244A, E or Q, specifically E190P, S244A, S244E and S244Q. Mutations at positions 275 and 279 are also of interest in combination with mutations at position 190.

The variants may additionally feature mutations at positions 1, 7, 118, 195, 202, 206, 321, 245 and 459, referring to SEQ ID NO: 1 for numbering, which are included in current, commercially-available α-amylases or proposed for such applications.

The variants may further include a deletion in the X1G/X2G₂ motif adjacent to the calcium-binding loop corresponding to R181, G182, T183, and G184, using SEQ ID NO: 1 for numbering. In some embodiments, the variant α-amylases include adjacent, pair-wise deletions of amino acid residues corresponding to R181 and G182, or T183 and G184. A deletion in amino acid residues corresponding to R181 and G182 may be referred to as “ΔRG,” while a deletion in amino acid residues corresponding to the residue at position 183 (usually T, D, or H) and G184 may be referred to as “ΔTG,” “ΔDG,” “ΔHG” etc., as appropriate. Both pair-wise deletions appear to produce the same effect in α-amylases.

The variants may further include previously described mutations for use in other α-amylases having a similar fold and/or having 60% or greater amino acid sequence identity to (i) any of the well-known Bacillus α-amylases, e.g., from B. lichenifomis (i.e., BLA and LAT), B. stearothermophilus (i.e., BSG), and B. amyloliquifaciens (i.e., P00692, BACAM, and BAA), or hybrids, thereof, (ii) any α-amylases catagorized as Carbohydrate-Active Enzymes database (CAZy) Family 13 α-amylases or (iii) any amylase that has heretofore been referred to by the descriptive term, “Termamyl-like.” Exemplary α-amylases include but are not limited to those from Bacillus sp. SG-1, Bacillus sp. 707, and α-amylases referred to as A7-7, SP722, DSM90 14 and KSM AP1378. Similarly, any of the combination of mutations described, herein, may produce performance advantages in these α-amylases, regardless of whether they have been described as maltopentaose/maltohexaose-producing α-amylases.

Specifically contemplated combinatorial variants are listed, below, using SEQ ID NO: 1 for numbering. As discussed, above, similar variants with ΔR183-ΔT184 instead of ΔR181-ΔG182 are expected to perform as well as those described in detail.
T40-S91-Q169-ΔR181-ΔG182-T183-H281
Q172-ΔR181-ΔG182-E190-S288
Q172-ΔR181-ΔG182-S244-S288-S474
S91-Q172-ΔR181-ΔG182-E190-1324
T40-S91-ΔR181-ΔG182-E190-F263
T40-S91-ΔR181-ΔG182-S244-Y364
S91-Q172-ΔR181-ΔG182-E190-1324
S91-W116-Q172-ΔR181-ΔG182-S244-H281-S288
T40-S91-Y100-W116-Q172-ΔR181-ΔG182-S244-H281
T40-S91-Q172-ΔR181-ΔG182-S244-F263-H281
S91-Q172-ΔR181-ΔG182-E190-1324
T40-S91-Q172-ΔR181-ΔG182-E190-H281-1324
Y364-ΔR181-ΔG182

In related α-amylases, including previous engineered α-amylases, the mutations may be described as:
X40-X91-X169-ΔR181-ΔG182-X183-X281
X172-ΔR181-ΔG182-X190-X288
X172-ΔR181-ΔG182-X244-X288-X474
X91-X172-ΔR181-ΔG182-X190-X324
X40-X91-ΔR181-ΔG182-X190-X263
X40-X91-ΔR181-ΔG182-X244-X364
X91-X172-ΔR181-ΔG182-X190-X324
X91-X116-X172-ΔR181-ΔG182-X244-X281-X288
X40-X91-X100-X116-X172-ΔR181-ΔG182-X244-X281
X40-X91-X172-ΔR181-ΔG182-X244-X263-X281
X91-X172-ΔR181-ΔG182-X190-X324
X40-X91-X172-ΔR181-ΔG182-X190-X281-X324
X364L-ΔR181-ΔG182

Such varants include those having two, three, four, five, six or more, of the following features: (a) D or N at position 40 and/or Rat position 91, and (b) F at position 100, Y at position 263, D at position 288, M, N or R at position 324 and/or L at position 364, optionally in combination with (c) H at position 169, M at position 183M, N or S at position 281, N or R at position 172, P at position 190, E, Q or R at position 244, R at position 474, R at postion 116, optionally in combination with pairwise deletions at positions 181 and 182 or 183 and 184.

The specific substitutions in the tested variants are listed below:
T40N-S91R-Q169H-ΔR181-ΔG182-T183M-H281N
Q172R-ΔR181-ΔG182-E190P-S288D
Q172R-ΔR181-ΔG182-S244E-S288D-5474R
S91R-Q172R-ΔR181-ΔG182-E190P-I324M
T40N-S91R-ΔR181-ΔG182-E190P-F263Y
T40N-S91R-ΔR181-ΔG182-S244E-Y364L
S91R-Q172R-ΔR181-ΔG182-E190P-I324R
S91R-W116R-Q172R-ΔR181-ΔG182-S244E-H281S-S288D
T40N-S91R-Y100E-W116R-Q172N-ΔR181-ΔG182-S244Q-H281S
T40N-S91R-Q172R-ΔR181-ΔG182-S244Q-F263Y-H281S
S91R-Q172R-ΔR181-ΔG182-E190P-I324N
T40D-S91R-Q172R-ΔR181-ΔG182-E190P-H281S-I324R
Y364L-ΔR181-ΔG182

It will be appreciated that where an α-amylase naturally has a mutation listed above (i.e., where the wild-type α-amylase already comprised a residue identified as a mutation), then that particular mutation does not apply to that molecule. However, other described mutations may work in combination with the naturally-occuring residue at that position.

The present variant α-amylases may also include the substitution, deletion or addition of one or several amino acids in the amino acid sequence, for example less than 10, less than 9, less than 8, less than 7, less than 6, less than 5, less than 4, less than 3, or even less than 2 substitutions, deletions or additions. Such variants are expected to have similar activity to the α-amylases from which they were derived. The present variant α-amylases may also include minor deletions and/or extensions of one or a few residues at their N or C-terminii. Such minor changes are unlikely to defeat the inventive conepts descibed, herein.

The present amylase may be “precursor,” “immature,” or “full-length,” in which case they include a signal sequence, or “mature,” in which case they lack a signal sequence. Mature forms of the polypeptides are generally the most useful. Unless otherwise noted, the amino acid residue numbering used herein refers to the mature forms of the respective amylase polypeptides.

In some embodiments, the variant α-amylase has at least 60%, at least 70%, at least 75%, at least 80%, at least 85%, at least 90%, at least 91%, at least 92%, at least 93%, at least 94%, at least 95%, at least 96%, at least 97%, at least 98%, or even at least 99%, but less than 100%, amino acid sequence identity to SEQ ID NO: 1, 2, 3 or 4.

### 2.5. Nucleotides Encoding Variant Amylase Polypeptides

In another aspect, nucleic acids encoding a variant α-amylase polypeptide are provided. The nucleic acid may encode a particular amylase polypeptide, or an α-amylase having a specified degree of amino acid sequence identity to the particular α-amylase.

In some embodiments, the nucleic acid encodes an α-amylase having at least 70%, at least 75%, at least 80%, at least 85%, at least 90%, at least 91%, at least 92%, at least 93%, at least 94%, at least 95%, at least 96%, at least 97%, at least 98%, or even at least 99%, but less than 100%, amino acid sequence identity to SEQ ID NO: 1, 2, 3 or 4. It will be appreciated that due to the degeneracy of the genetic code, a plurality of nucleic acids may encode the same polypeptide.

In some embodiments, the nucleic acid hybridizes under stringent or very stringent conditions to a nucleic acid encoding (or complementary to a nucleic acid encoding) an α-amylase having at least 70%, at least 75%, at least 80%, at least 85%, at least 90%, at least 91%, at least 92%, at least 93%, at least 94%, at least 95%, at least 96%, at least 97%, at least 98%, or even at least 99%, but less than 100%, amino acid sequence identity to SEQ ID NO: 1, 2, 3 or 4.

### 3. Production of Variant α-Amylases

The present variant α-amylases can be produced in host cells, for example, by secretion or intracellular expression, using methods well-known in the art. Fermentation, separation, and concentration techniques are well known in the art and conventional methods can be used to prepare a concentrated, variant-α-amylase-polypeptide-containing solution.

For production scale recovery, variant α-amylase polypeptides can be enriched or partially purified as generally described above by removing cells via flocculation with polymers. Alternatively, the enzyme can be enriched or purified by microfiltration followed by concentration by ultrafiltration using available membranes and equipment. However, for some applications, the enzyme does not need to be enriched or purified, and whole broth culture can be lysed and used without further treatment. The enzyme can then be processed, for example, into granules.

### 4. Cleaning Compositions Containing Variant α-Amylases

An aspect of the present compositions and methods involves a cleaning composition that includes a variant α-amylase as a component for, e.g., automatic and manual dishwashing (ADW), laundry washing, and other hard-surface cleaning.

### 4.1. Overview

Preferably, the variant α-amylase is incorporated into detergent formulations at or below the concentration conventionally used for known α-amylases. Because the described α-amylase variants are superior in performance to any previously available, they are expected to deliver superior perfomance at standard doses, and similar performance at lower doses, compared to existing α-amylases. Particular forms and formulations of detergent compositions for inclusion of the present α-amylase are described, below.

### 4.2. Automatic Dishwashing (ADW) Detergent Composition

Exemplary ADW detergent compositions include non-ionic surfactants, including ethoxylated non-ionic surfactants, alcohol alkoxylated surfactants, epoxy-capped poly(oxyalkylated) alcohols, or amine oxide surfactants present in amounts from 0 to 10% by weight; builders in the range of 5-60% including phosphate builders (e.g., mono-phosphates, di-phosphates, tri-polyphosphates, other oligomeric-poylphosphates, sodium tripolyphosphate-STPP) and phosphate-free builders (e.g., amino acid-based compounds including methyl-glycine-diacetic acid (MGDA) and salts and derivatives thereof, glutamic-N,N-diacetic acid (GLDA) and salts and derivatives thereof, iminodisuccinic acid (IDS) and salts and derivatives thereof, carboxy methyl inulin and salts and derivatives thereof, nitrilotriacetic acid (NTA), diethylene triamine pentaacetic acid (DTPA), β-alaninediacetic acid (β-ADA) and their salts, homopolymers and copolymers of poly-carboxylic acids and their partially or completely neutralized salts, monomeric polycarboxylic acids and hydroxycarboxylic acids and their salts in the range of 0.5% to 50% by weight; sulfonated/carboxylated polymers in the range of about 0.1% to about 50% by weight to provide dimensional stability; drying aids in the range of about 0.1% to about 10% by weight (e.g., polyesters, especially anionic polyesters, optionally together with further monomers with 3 to 6 functionalities—typically acid, alcohol or ester functionalities which are conducive to polycondensation, polycarbonate-, polyurethane- and/or polyurea-polyorganosiloxane compounds or precursor compounds, thereof, particularly of the reactive cyclic carbonate and urea type); silicates in the range from about 1% to about 20% by weight (including sodium or potassium silicates for example sodium disilicate, sodium meta-silicate and crystalline phyllosilicates); inorganic bleach (e.g., perhydrate salts such as perborate, percarbonate, perphosphate, persulfate and persilicate salts) and organic bleach (e.g., organic peroxyacids, including diacyl and tetraacylperoxides, especially diperoxydodecanedioc acid, diperoxytetradecanedioc acid, and diperoxyhexadecanedioc acid); bleach activators (i.e., organic peracid precursors in the range from about 0.1% to about 10% by weight); bleach catalysts (e.g., manganese triazacyclononane and related complexes, Co, Cu, Mn, and Fe bispyridylamine and related complexes, and pentamine acetate cobalt(III) and related complexes); metal care agents in the range from about 0.1% to 5% by weight (e.g., benzatriazoles, metal salts and complexes, and/or silicates); enzymes in the range from about 0.01 to 5.0 mg of active enzyme per gram of automatic dishwashing detergent composition (e.g., proteases, α-amylases, lipases, cellulases, choline oxidases, peroxidases/oxidases, pectate lyases, mannanases, cutinases, laccases, phospholipases, lysophospholipases, acyltransferase, perhydrolase, arylesterase, and mixtures thereof); and enzyme stabilizer components (e.g., oligosaccharides, polysaccharides, and inorganic divalent metal salts).

A particular exemplary ADW composition in which at least some of the present variants have been tested is shown in Table 2.

TABLE 2
Exemplary ADW composition

| Ingredient | Weight in grams |
|---|---|
| Bleach Activator (tetraacetylethylenediamine; | 0.22 |
| TAED) | |
| SKS-6 sodium disilicate (Na₂Si₂O₅) | 0.8 |
| hydroxy-ethane diphosphonic acid (HEDP) | 0.93 |
| Sodium carbonate | 1.5 |
| MGDA | 7.01 |
| Sulfonic acid group-containing polymer | 0.80 |
| (Acusol ™ 588) | |
| Sodium percarbonate | 3.50 |
| Bleach catalyst (Manganese 1,4,7- | 0.256 |
| triazacyclononane; MnTACN) | |
| LUTENSOL ® TO7 | 0.90 |
| PLURAFAC ® SLF 180 | 0.75 |
| Dipropylene glycol | 0.40 |
| Minor components | balance |
| Total % of full dose | 100 |

### 4.3. Heavy Duty Liquid (HDL) Laundry Detergent Composition

Exemplary HDL laundry detergent compositions includes a detersive surfactant (10%-40% wt/wt), including an anionic detersive surfactant (selected from a group of linear or branched or random chain, substituted or unsubstituted alkyl sulphates, alkyl sulphonates, alkyl alkoxylated sulphate, alkyl phosphates, alkyl phosphonates, alkyl carboxylates, and/or mixtures thereof), and optionally non-ionic surfactant (selected from a group of linear or branched or random chain, substituted or unsubstituted alkyl alkoxylated alcohol, for example a C8-C18 alkyl ethoxylated alcohol and/or C6-C12 alkyl phenol alkoxylates), wherein the weight ratio of anionic detersive surfactant (with a hydrophilic index (HIc) of from 6 to 9) to non-ionic detersive surfactant is greater than 1:1. Suitable detersive surfactants also include cationic detersive surfactants (selected from a group of alkyl pyridinium compounds, alkyl quarternary ammonium compounds, alkyl quarternary phosphonium compounds, alkyl ternary sulphonium compounds, and/or mixtures thereof); zwitterionic and/or amphoteric detersive surfactants (selected from a group of alkanolamine sulpho-betaines); ampholytic surfactants; semi-polar non-ionic surfactants and mixtures thereof.

The composition may optionally include, a surfactancy boosting polymer consisting of amphiphilic alkoxylated grease cleaning polymers (selected from a group of alkoxylated polymers having branched hydrophilic and hydrophobic properties, such as alkoxylated polyalkylenimines in the range of 0.05 wt % to 10 wt %) and/or random graft polymers (typically comprising of hydrophilic backbone comprising monomers selected from the group consisting of: unsaturated C1-C6 carboxylic acids, ethers, alcohols, aldehydes, ketones, esters, sugar units, alkoxy units, maleic anhydride, saturated polyalcohols such as glycerol, and mixtures thereof; and hydrophobic side chain(s) selected from the group consisting of: C4-C25 alkyl group, polypropylene, polybutylene, vinyl ester of a saturated C1-C6 mono-carboxylic acid, C1-C6 alkyl ester of acrylic or methacrylic acid, and mixtures thereof.

The composition may include additional polymers such as soil release polymers (include anionically end-capped polyesters, for example SRP1, polymers comprising at least one monomer unit selected from saccharide, dicarboxylic acid, polyol and combinations thereof, in random or block configuration, ethylene terephthalate-based polymers and co-polymers thereof in random or block configuration, for example Repel-o-tex SF, SF-2 and SRP6, Texcare SRA100, SRA300, SRN100, SRN170, SRN240, SRN300 and SRN325, Marloquest SL), anti-redeposition polymers (0.1 wt % to 10 wt %, include carboxylate polymers, such as polymers comprising at least one monomer selected from acrylic acid, maleic acid (or maleic anhydride), fumaric acid, itaconic acid, aconitic acid, mesaconic acid, citraconic acid, methylenemalonic acid, and any mixture thereof, vinylpyrrolidone homopolymer, and/or polyethylene glycol, molecular weight in the range of from 500 to 100,000 Da); cellulosic polymer (including those selected from alkyl cellulose, alkyl alkoxyalkyl cellulose, carboxyalkyl cellulose, alkyl carboxyalkyl cellulose examples of which include carboxymethyl cellulose, methyl cellulose, methyl hydroxyethyl cellulose, methyl carboxymethyl cellulose, and mixures thereof) and polymeric carboxylate (such as maleate/acrylate random copolymer or polyacrylate homopolymer).

The composition may further include saturated or unsaturated fatty acid, preferably saturated or unsaturated C12-C24 fatty acid (0 wt % to 10 wt %); deposition aids (examples for which include polysaccharides, preferably cellulosic polymers, poly diallyl dimethyl ammonium halides (DADMAC), and co-polymers of DAD MAC with vinyl pyrrolidone, acrylamides, imidazoles, imidazolinium halides, and mixtures thereof, in random or block configuration, cationic guar gum, cationic cellulose such as cationic hydoxyethyl cellulose, cationic starch, cationic polyacylamides, and mixtures thereof.

The composition may further include dye transfer inhibiting agents, examples of which include manganese phthalocyanine, peroxidases, polyvinylpyrrolidone polymers, polyamine N-oxide polymers, copolymers of N-vinylpyrrolidone and N-vinylimidazole, polyvinyloxazolidones and polyvinylimidazoles and/or mixtures thereof; chelating agents, examples of which include ethylene-diamine-tetraacetic acid (EDTA), diethylene triamine penta methylene phosphonic acid (DTPMP), hydroxy-ethane diphosphonic acid (HEDP), ethylenediamine N,N′-disuccinic acid (EDDS), methyl glycine diacetic acid (MGDA), diethylene triamine penta acetic acid (DTPA), propylene diamine tetracetic acid (PDTA), 2-hydroxypyridine-N-oxide (HPNO), or methyl glycine diacetic acid (MGDA), glutamic acid N,N-diacetic acid (N,N-dicarboxymethyl glutamic acid tetrasodium salt (GLDA), nitrilotriacetic acid (NTA), 4,5-dihydroxy-m-benzenedisulfonic acid, citric acid and any salts thereof, N-hydroxyethylethylenediaminetri-acetic acid (HEDTA), triethylenetetraaminehexaacetic acid (TTHA), N-hydroxyethyliminodiacetic acid (HEIDA), dihydroxyethylglycine (DHEG), ethylenediaminetetrapropionic acid (EDTP), and derivatives thereof.

The composition preferably included enzymes (generally about 0.01 wt % active enzyme to 0.03 wt % active enzyme) selected from proteases, α-amylases, lipases, cellulases, choline oxidases, peroxidases/oxidases, pectate lyases, mannanases, cutinases, laccases, phospholipases, lysophospholipases, acyltransferases, perhydrolases, arylesterases, and any mixture thereof. The composition may include an enzyme stabilizer (examples of which include polyols such as propylene glycol or glycerol, sugar or sugar alcohol, lactic acid, reversible protease inhibitor, boric acid, or a boric acid derivative, e.g., an aromatic borate ester, or a phenyl boronic acid derivative such as 4-formylphenyl boronic acid).

The composition optionally includes silicone or fatty-acid based suds suppressors; hueing dyes, calcium and magnesium cations, visual signaling ingredients, anti-foam (0.001 wt % to about 4.0 wt %), and/or structurant/thickener (0.01 wt % to 5 wt %, selected from the group consisting of diglycerides and triglycerides, ethylene glycol distearate, microcrystalline cellulose, cellulose based materials, microfiber cellulose, biopolymers, xanthan gum, gellan gum, and mixtures thereof).

The composition can be any liquid form, for example a liquid or gel form, or any combination thereof. The composition may be in any unit dose form, for example a pouch.

### 4.4. Heavy Duty Dry/Solid (HDD) Laundry Detergent Composition

Exemplary HDD laundry detergent compositions includes a detersive surfactant, including anionic detersive surfactants (e.g., linear or branched or random chain, substituted or unsubstituted alkyl sulphates, alkyl sulphonates, alkyl alkoxylated sulphate, alkyl phosphates, alkyl phosphonates, alkyl carboxylates and/or mixtures thereof), non-ionic detersive surfactant (e.g., linear or branched or random chain, substituted or unsubstituted C8-C18 alkyl ethoxylates, and/or C6-C12 alkyl phenol alkoxylates), cationic detersive surfactants (e.g., alkyl pyridinium compounds, alkyl quaternary ammonium compounds, alkyl quaternary phosphonium compounds, alkyl ternary sulphonium compounds, and mixtures thereof), zwitterionic and/or amphoteric detersive surfactants (e.g., alkanolamine sulpho-betaines), ampholytic surfactants, semi-polar non-ionic surfactants, and mixtures thereof; builders including phosphate free builders (for example zeolite builders examples which include zeolite A, zeolite X, zeolite P and zeolite MAP in the range of 0 wt % to less than 10 wt %), phosphate builders (for example sodium tri-polyphosphate in the range of 0 wt % to less than 10 wt %), citric acid, citrate salts and nitrilotriacetic acid, silicate salt (e.g., sodium or potassium silicate or sodium meta-silicate in the range of 0 wt % to less than 10 wt %, or layered silicate (SKS-6)); carbonate salt (e.g., sodium carbonate and/or sodium bicarbonate in the range of 0 wt % to less than 80 wt %); and bleaching agents including photobleaches (e.g., sulfonated zinc phthalocyanines, sulfonated aluminum phthalocyanines, xanthenes dyes, and mixtures thereof) hydrophobic or hydrophilic bleach activators (e.g., dodecanoyl oxybenzene sulfonate, decanoyl oxybenzene sulfonate, decanoyl oxybenzoic acid or salts, thereof, 3,5,5-trimethy hexanoyl oxybenzene sulfonate, tetraacetyl ethylene diamine-TAED, nonanoyloxybenzene sulfonate-NOBS, nitrile quats, and mixtures thereof), sources of hydrogen peroxide (e.g., inorganic perhydrate salts examples of which include mono or tetra hydrate sodium salt of perborate, percarbonate, persulfate, perphosphate, or persilicate), preformed hydrophilic and/or hydrophobic peracids (e.g., percarboxylic acids and salts, percarbonic acids and salts, perimidic acids and salts, peroxymonosulfuric acids and salts, and mixtures thereof), and/or bleach catalysts (e.g., imine bleach boosters (examples of which include iminium cations and polyions), iminium zwitterions, modified amines, modified amine oxides, N-sulphonyl imines, N-phosphonyl imines, N-acyl imines, thiadiazole dioxides, perfluoroimines, cyclic sugar ketones, and mixtures thereof, and metal-containing bleach catalysts (e.g., copper, iron, titanium, ruthenium, tungsten, molybdenum, or manganese cations along with an auxiliary metal cations such as zinc or aluminum and a sequestrate such as ethylenediaminetetraacetic acid, ethylenediaminetetra(methylenephosphonic acid), and water-soluble salts, thereof).

The composition preferably includes enzymes, e.g., proteases, α-amylases, lipases, cellulases, choline oxidases, peroxidases/oxidases, pectate lyases, mannanases, cutinases, laccases, phospholipases, lysophospholipases, acyltransferase, perhydrolase, arylesterase, and any mixture thereof.

The composition may optionally include additional detergent ingredients including perfume microcapsules, starch encapsulated perfume accord, hueing agents, additional polymers, including fabric integrity and cationic polymers, dye-lock ingredients, fabric-softening agents, brighteners (for example C.I. Fluorescent brighteners), flocculating agents, chelating agents, alkoxylated polyamines, fabric deposition aids, and/or cyclodextrin.

### 4.5. Additional Enzymes

Any of the cleaning compositions described, herein, may include any number of additional enzymes. In general, the enzyme(s) should be compatible with the selected detergent, (e.g., with respect to pH-optimum, compatibility with other enzymatic and non-enzymatic ingredients, and the like), and the enzyme(s) should be present in effective amounts. The following enzymes are provided as examples.

### Proteases:

Suitable proteases include those of animal, vegetable or microbial origin. Chemically modified or protein engineered mutants are included, as well as naturally processed proteins. The protease may be a serine protease or a metalloprotease, an alkaline microbial protease, a trypsin-like protease, or a chymotrypsin-like protease. Examples of alkaline proteases are subtilisins, especially those derived from Bacillus, e.g., subtilisin Novo, subtilisin Carlsberg, subtilisin 309, subtilisin 147, and subtilisin 168 (see, e.g., WO 1989/06279). Exemplary proteases include but are not limited to those described in WO 1995/23221, WO 1992/21760, WO 2008/010925, WO 2010/0566356, WO 2011/072099, WO 2011/13022, WO 2011/140364, WO 2012/151534, WO 2015/038792, WO 2015/089441, WO 2015/089447, WO 2015/143360, WO 2016/001449, WO 2016/001450, WO 2016/061438, WO 2016/069544, WO 2016/069548, WO 2016/069552, WO 2016/069557, WO 2016/069563, WO 2016/069569, WO 2016/087617, WO 2016/087619, WO 2016/145428, WO 2016/174234, WO 2016/183509, WO 2016/202835, WO 2016/205755, WO 2008/0090747, WO 2018/118950, WO 2018/169750, WO/2018/118917, U.S. Pat. Nos. 5,801,039, 5,340,735, 5,500,364, 5,855,625, RE 34606, U.S. Pat. Nos. 5,955,340, 5,700,676, 6,312,936, 6,482,628, 8,530,219, as well as metalloproteases described in WO 2007/044993, WO 2009/058303, WO 2009/058661, WO 2014/071410, WO 2014/194032, WO 2014/194034, WO 2014/194054, and WO 2014/194117.

Exemplary commercial proteases include, but are not limited to MAXATASE, MAXACAL, MAXAPEM, OPTICLEAN®, OPTIMASE®, PROPERASE®, PURAFECT®, PURAFECT® OXP, PURAMAX®, EXCELLASE®, PREFERENZ™ proteases (e.g., P100, P110, P280, P300), EFFECTENZ™ proteases (e.g., P1000, P1050, P2000), EXCELLENZ™ proteases (e.g., P1000), ULTIMASE®, and PURAFAST (Danisco US); ALCALASE®, ALCALASE® ULTRA, BLAZE®, BLAZE® EVITY®, BLAZE® EVITY® 16L, CORONASE®, SAVINASE®, SAVINASE® ULTRA, SAVINASE® EVITY®, SAVINASE® EVERTS®, PRIMASE, DURAZYM, POLARZYME®, OVOZYME®, KANNASE®, LIQUANASE®, EVERTS®, NEUTRASE®, PROGRESS UNO®, RELASE® and ESPERASE® (Novozymes); BLAP™ and BLAP™ variants (Henkel); LAVERGY™ PRO 104 L (BASF), and KAP® (B. alkalophilus subtilisin) (Kao). Suitable proteases include naturally occurring proteases or engineered variants specifically selected or engineered to work at relatively low temperatures.

In particular embodiments of the present compositions and methods, the described α-amylase variants are used in combination with a variant subtilisin protease from Bacillus gibsonii (referred to as BG46) having the amino acid substitutions X39E, X99R, X126A, X127E and X128G, and further having one or more additional substitutions selected from the group consisting of N74D-M211L-N253P, R179Q-M211L-N253P, N74D-N253P, N85R-G160Q-R179Q-M211L-N212S-N253P, R179Q-N253P, G160Q-R179Q-M211L-N212S-N253P, R179Q-M211L, G160Q-R179Q-M211L-N253P, G160Q-R179Q-N212S-N253P, N74D-M211L, M211L-N242D, G160Q-R179Q-M211L-N212S, N74D-R179Q-M211L-N253P, G160Q-R179Q-M211L, G160Q-R179Q-N253P, N74D-Q200L-M211L, N74D-G160Q-N212S-N253P, N74D-G160Q-M211L-N253P, G160Q-R179Q, G160Q-R179Q-N212S, N74D-G160Q-N253P, N74D-G160Q-R179Q-M211L-N212S-N253P, N74D-N085R-G160Q-R179Q-M211L, N74D-G160Q-M211L-N212S-N253P, N74D-N085R-N116R-Q200L-Q256E, N74D-G160Q-R179Q-N212S-N253P, N74D-G160Q-M211L-N212S, N74D-G160Q, N74D-G160Q-R179Q-M211L-N253P, N74D-R179Q-M211L, N74D-G160Q-N212S, N74D-G160Q-M211L, N74D-G160Q-R179Q-N253P, N74D, N74D-G160Q-R179Q-M211L-N212S, N74D-N085R-M211L-N212S, N74D-G160Q-R179Q-N212S, N74D-G160Q-R179Q-M211L, N74D-M211L-Q256E, N74D-G160Q-R179Q, R179Q-M211L-N212S-N253P, R179Q-M211L-N212S, N74D-N085R-R179Q-M211L-N212S, N74D-M211L-N212S, N74D-R179Q-M211L-N212S, N74D-M211L-N242D, N74D-Q200L-M211L-Q256E, N74D-Q200L-M211L-N242D-Q256E, N74D-Q200L, N74D-M211N-N212Q, N74D-M211N-N212Q-Q256E, N74D-M211N-Q256E, N74D-M211Q, N74D-M211Q-N212Q, N74D-M211Q-N212Q-Q256E, N74D-M211Q-Q256E, N74D-N198A-M211Q, N74D-N198A-M211Q-N212Q, N74D-N198A-M211Q-Q256E, N74D-N198G-M211Q, N74D-N198G-M211Q-N212Q, N74D-N198G-M211Q-Q256E, N74D-N198K-M211Q-N212Q, N74D-N198L-M211Q-N212Q, N74D-N198Q-M211Q-N212Q, N74D-N198R-M211Q-N212Q, N74D-N198T-M211Q-N212Q, N74D-N198V-M211Q-N212Q, N74D-N212Q-Q256E, N74D-Q256E, N74D-R207Q, N74D-R207Q-M211N, N74D-R207Q-M211N-N212Q, N74D-R207Q-M211N-N212Q-Q256E, N74D-R207Q-M211N-Q256E, N74D-R207Q-M211Q, N74D-R207Q-M211Q-N212Q, N74D-R207Q-M211Q-N212Q-Q256E, N74D-R207Q-N212Q, N74D-R207Q-N212Q-Q256E, N74D-R207Q-Q256E, N74D-N198S-M211Q and N74D-N198L-M211Q, wherein the amino acid positions are numbered by correspondence with the amino acid sequence of SEQ ID NO: 5, wherein the variant has at least 90% identity to amino acid sequence identity to the amino acid sequence of SEQ ID NO: 6. These amino acid sequences are shown, below.

Amino acid sequence of BG46 protease (SEQ ID NO: 5):

QQTVPWGITRVQAPAVHNRGITGSGVRVAILDSGISAHSDLNIRGGASF
VPGEPTTADLNGHGTHVAGTVAALNNSIGVIGVAPNAELYAVKVLGANG
SGSVSGIAQGLEWAATNNMHIANMSLGSDFPSSTLERAVNYATSRDVLV
IAATGNNGSGSVGYPARYANAMAVGATDQNNRRANFSQYGTGIDIVAPG
VNVQSTYPGNRYVSMNGTSMATPHVAGAAALVKQRYPSWNATQIRNHLK
NTATNLGNSSQFGSGLVNAEAATR

Amino acid sequence of BG46 with the substitutions S39E, S99R, S126A, D127E and F128G (SEQ ID NO: 6):

QQTVPWGITRVQAPAVHNRGITGSGVRVAILDSGISAHEDLNIRGGASF
VPGEPTTADLNGHGTHVAGTVAALNNSIGVIGVAPNAELYAVKVLGANG
RGSVSGIAQGLEWAATNNMHIANMSLGAEGPSSTLERAVNYATSRDVLV
IAATGNNGSGSVGYPARYANAMAVGATDQNNRRANFSQYGTGIDIVAPG
VNVQSTYPGNRYVSMNGTSMATPHVAGAAALVKQRYPSWNATQIRNHLK
NTATNLGNSSQFGSGLVNAEAATR

### Lipases:

Suitable lipases include those of bacterial or fungal origin. Chemically modified, proteolytically modified, or protein engineered mutants are included. Examples of useful lipases include but are not limited to lipases from Humicola (synonym Thermomyces), e.g., from H lanuginosa (T lanuginosus) (see, e.g., EP 258068 and EP 305216), from H. insolens (see, e.g., WO 96/13580); a Pseudomonas lipase (e.g., from P. alcaligenes or P. pseudoalcaligenes; see, e.g., EP 218 272), P. cepacia (see, e.g., EP 331 376), P. stutzeri (see e.g., GB 1,372,034), P. fluorescens, Pseudomonas sp. strain SD 705 (see, e.g., WO 95/06720 and WO 96/27002), P. wisconsinensis (see, e.g., WO 96/12012); a Bacillus lipase (e.g., from B. subtilis; see e.g., Dartois et al. (1993)Biochemica et Biophysica Acta 1131:253-360), B. stearothermophilus (see, e.g., JP 64/744992), or B. pumilus (see, e.g., WO 91/16422). Additional lipase variants contemplated for use in the formulations include those described for example in: WO 92/05249, WO 94/01541, WO 95/35381, WO 96/00292, WO 95/30744, WO 94/25578, WO 95/14783, WO 95/22615, WO 97/04079, WO 97/07202, EP 407225, and EP 260105.

Exemplary commercial lipases include, but are not limited to M1 LIPASE, LUMA FAST, and LIPOMAX (Genencor); LIPEX®, LIPOCLEAN®, LIPOLASE® and LIPOLASE® ULTRA (Novozymes); and LIPASE P (Amano Pharmaceutical Co. Ltd).

### Polyesterases:

Suitable polyesterases can be included in the composition, such as those described in, for example, WO 01/34899, WO 01/14629, and U.S. Pat. No. 6,933,140.

### Amylases:

The present compositions can be combined with other amylases, including other α-amylases. Such a combination is particularly desirable when different α-amylases demonstrate different performance characteristics and the combination of a plurality of different α-amylases results in a composition that provides the benefits of the different α-amylases. Other α-amylases include commercially available α-amylases, such as but not limited to STAINZYME®, NATALASE®, DURAMYL®, TERMAMYL®, FUNGAMYL® and BAN™ (Novo Nordisk A/S and Novozymes A/S); RAPIDASE®, POWERASE®, PURASTAR®, and PREFERENZ™ (from DuPont Industrial Biosciences). Exemplary α-amylases are described in WO 94/18314A1, WO 2008/0293607, WO 2013/063460, WO 10/115028, WO 2009/061380A2, WO 2014/099523, WO 2015/077126A1, WO 2013/184577, WO 2014/164777, WO 95/10603, WO 95/26397, WO 96/23874, WO 96/23873, WO 97/41213, WO 99/19467, WO 00/60060, WO 00/29560, WO 99/23211, WO 99/46399, WO 00/60058, WO 00/60059, WO 99/42567, WO 01/14532, WO 02/092797, WO 01/66712, WO 01/88107, WO 01/96537, WO 02/10355, WO 2006/002643, WO 2004/055178, and WO 98/13481.

### Cellulases:

Cellulases can be added to the compositions. Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed for example in U.S. Pat. Nos. 4,435,307; 5,648,263; 5,691,178; 5,776,757; and WO 89/09259. Exemplary cellulases contemplated for use are those having color care benefit for the textile. Examples of such cellulases are cellulases described in for example EP 0495257, EP 0531372, WO 96/11262, WO 96/29397, and WO 98/08940. Other examples are cellulase variants, such as those described in WO 94/07998; WO 98/12307; WO 95/24471; PCT/DK98/00299; EP 531315; U.S. Pat. Nos. 5,457,046; 5,686,593; and 5,763,254. Exemplary cellulases include those described in WO2005054475, WO2005056787, U.S. Pat. Nos. 7,449,318, 7,833,773, 4,435,307; EP 0495257; and U.S. Provisional Appl. Nos. 62/296,678 and 62/435,340. Exemplary commercial cellulases include, but are not limited to, CELLUCLEAN®, CELLUZYME®, CAREZYME®, CAREZYME® PREMIUM, ENDOLASE®, and RENOZYME® (Novozymes), REVITALENZ®100, REVITALENZ® 200/220 and REVITALENZ® 2000 (Danisco US); BIOTOUCH® (AB Enzymes) and KAC-500(B) (Kao Corporation).

### Mannanases:

Exemplary mannanases include, but are not limited to, those of bacterial or fungal origin, such as, for example, as is described in WO2016007929; USPNs 6566114, 6602842, and 6440991; and International Appl Nos. PCT/US2016/060850 and PCT/US2016/060844. Exemplary mannanases include, but are not limited to, those of bacterial or fungal origin, such as, for example, as is described in WO2016007929; USPNs 6566114, 6602842, and 6440991; and International Appl Nos. PCT/US2016/060850 and PCT/US2016/060844.

### Peroxidases/Oxidases:

Suitable peroxidases/oxidases contemplated for use in the compositions include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257. Commercially available peroxidases include for example GUARDZYME™ (Novo Nordisk A/S and Novozymes A/S).

The detergent composition can also comprise 2,6-β-D-fructan hydrolase, which is effective for removal/cleaning of biofilm present on household and/or industrial textile/laundry.

The detergent enzyme(s) may be included in a detergent composition by adding separate additives containing one or more enzymes, or by adding a combined additive comprising all of these enzymes. A detergent additive, i.e. a separate additive or a combined additive, can be formulated, e.g., as a granulate, a liquid, a slurry, and the like. Exemplary detergent additive formulations include but are not limited to granulates, in particular non-dusting granulates, liquids, in particular stabilized liquids or slurries.

### Perhydrolases:

Perhydrolases include those described in, for example, WO2005/056782, WO2007/106293, WO 2008/063400, WO2008/106214 and WO2008/106215.

### Nucleases:

Suitable nucleases include, but are not limited to, those described in WO2015/181287, WO2015/155350, WO2016/162556, WO2017/162836, WO2017/060475 (e.g. SEQ ID NO: 21), WO2018/184816, WO2018/177936, WO2018/177938, WO2018/185269, WO2018/185285, WO2018/177203, WO2018/184817, WO2019/084349, WO2019/084350, WO2019/081721, WO2018/076800, WO2018/185267, WO2018/185280, and WO2018/206553.

Other nucleases that can be used in combination with the present variant α-amylases include those described in Nijland, R. et al. (2010) PLoS ONE 5-e15668 and Whitchurch, C. B. et al. (2002) Science 295:1487.

### 4.6. Forms of Cleaning Compositions

The detergent composition may be in any convenient form, e.g., a bar, a tablet, a powder, a granule, a paste, or a liquid. A liquid detergent may be aqueous, typically containing up to about 70% water, and 0% to about 30% organic solvent. Compact detergent gels containing about 30% or less water are also contemplated. The present variant α-amylase are compatible with known forms and formulations of detergent compositions and particular forms and formulations are described, herein.

Numerous exemplary detergent formulations to which the present α-amylases can be added (or is in some cases are identified as a component of) are described in WO2013063460. These include commercially available unit dose detergent formulations/packages such as PUREX® UltraPacks (Henkel), FINISH® Quantum (Reckitt Benckiser), CLOROX™ 2 Packs (Clorox), OxiClean Max Force Power Paks (Church & Dwight), TIDE® Stain Release, TIDE® Pods, CASCADE® ActionPacs, CASCADE® Platimun, CASCADE® and Pure essential, (Procter & Gamble). Unit dose formulations and packaging are described in, for example, US20090209445A1, US20100081598A1, U.S. Pat. No. 7,001,878B2, EP1504994B1, WO2001085888A2, WO2003089562A1, WO2009098659A1, WO2009098660A1, WO2009112992A1, WO2009124160A1, WO2009152031A1, WO2010059483A1, WO2010088112A1, WO2010090915A1, WO2010135238A1, WO2011094687A1, WO2011094690A1, WO2011127102A1, WO2011163428A1, WO2008000567A1, WO2006045391A1, WO2006007911A1, WO2012027404A1, EP1740690B1, WO2012059336A1, U.S. Pat. No. 6,730,646B1, WO2008087426A1, WO2010116139A1, and WO2012104613A1.

### 5. Carbohydrate Processing Using Variant α-Amylases

The variant α-amylases may be useful for a variety of industrial carbohydrate processing applications. For example, the variant α-amylases may be useful in a starch conversion process, particularly in a saccharification process of a starch that has undergone liquefaction. The desired end-product may be any product that may be produced by the enzymatic conversion of the starch substrate. For example, the desired product may be a syrup rich in glucose and maltose, which can be used in other processes, such as the preparation of HFCS, or which can be converted into a number of other useful products, such as ascorbic acid intermediates (e.g., gluconate; 2-keto-L-gulonic acid; 5-keto-gluconate; and 2,5-diketogluconate); 1,3-propanediol; aromatic amino acids (e.g., tyrosine, phenylalanine and tryptophan); organic acids (e.g., lactate, pyruvate, succinate, isocitrate, and oxaloacetate); amino acids (e.g., serine and glycine); antibiotics; antimicrobials; enzymes; vitamins; and hormones.

The starch conversion process may be a precursor to, or simultaneous with, a fermentation process designed to produce alcohol for fuel or drinking (i.e., potable alcohol). One skilled in the art is aware of various fermentation conditions that may be used in the production of these end-products. Variant α-amylases are also useful in compositions and methods of food preparation. These various uses of variant α-amylases are described in more detail below.

### 5.1. Preparation of Starch Substrates

Methods for preparing starch substrates for use in the processes disclosed herein are well known. Useful starch substrates may be obtained from, e.g., tubers, roots, stems, legumes, cereals or whole grain. More specifically, the granular starch may be obtained from corn, cobs, wheat, barley, rye, triticale, milo, sago, millet, cassava, tapioca, sorghum, rice, peas, bean, banana, or potatoes. Specifically contemplated starch substrates are corn starch and wheat starch. The starch from a grain may be ground or whole and includes corn solids, such as kernels, bran and/or cobs. The starch may also be highly refined raw starch or feedstock from starch refinery processes.

### 5.2. Gelatinization and Liquefaction of Starch

Gelatinization is generally performed simultaneously with, or followed by, contacting a starch substrate with an α-amylase, although additional liquefaction-inducing enzymes optionally may be added. In some embodiments, the starch substrate prepared as described above is slurried with water. To optimize α-amylase stability and activity, the pH of the slurry typically is adjusted to about pH 4.5-6.5 and about 1 mM of calcium (about 40 ppm free calcium ions) can also be added, depending upon the properties of the variant α-amylase used. α-amylase remaining in the slurry following liquefaction may be deactivated via a number of methods, including lowering the pH in a subsequent reaction step or by removing calcium from the slurry in cases where the enzyme is dependent upon calcium. The slurry of starch plus the α-amylase may be pumped continuously through a jet cooker, which is steam heated to 105° C. The slurry is then allowed to cool to room temperature.

### 5.3. Saccharification

The liquefied starch can be saccharified into a syrup that is rich in lower DP (e.g., DP1+DP2) saccharides, using variant α-amylases, optionally in the presence of another enzyme(s). The exact composition of the products of saccharification depends on the combination of enzymes used, as well as the type of granular starch processed.

Whereas liquefaction is generally run as a continuous process, saccharification is often conducted as a batch process. Saccharification typically is most effective at temperatures of about 60-65° C. and a pH of about 4.0-4.5, e.g., pH 4.3, necessitating cooling and adjusting the pH of the liquefied starch. Saccharification is normally conducted in stirred tanks, which may take several hours to fill or empty. Enzymes typically are added either at a fixed ratio to dried solids as the tanks are filled or added as a single dose at the commencement of the filling stage. A saccharification reaction to make a syrup typically is run over about 24-72 hours, for example, 24-48 hours. When a maximum or desired DE has been attained, the reaction is stopped by heating to 85° C. for 5 min., for example. Further incubation will result in a lower DE, eventually to about 90 DE, as accumulated glucose re-polymerizes to isomaltose and/or other reversion products via an enzymatic reversion reaction and/or with the approach of thermodynamic equilibrium.

### 5.4. Isomerization

The soluble starch hydrolysate produced by treatment with the variant α-amylase can be converted into high fructose starch-based syrup (HFSS), such as high fructose corn syrup (HFCS). This conversion can be achieved using a glucose isomerase, particularly a glucose isomerase immobilized on a solid support. The pH is increased to about 6.0 to about 8.0, e.g., pH 7.5 (depending on the isomerase), and Ca²⁺ is removed by ion exchange. Suitable isomerases include SWEETZYME®, IT (Novozymes A/S); G-ZYME® IMGI, and G-ZYME® G993, KETOMAX®, G-ZYME® G993, G-ZYME® G993 liquid, and GENSWEET® IGI. Following isomerization, the mixture typically contains about 40-45% fructose, e.g., 42% fructose.

### 5.5. Fermentation

The soluble starch hydrolysate, particularly a glucose rich syrup, can be fermented by contacting the starch hydrolysate with a fermenting organism typically at a temperature around 32° C., such as from 30° C. to 35° C. for alcohol-producing yeast. The temperature and pH of the fermentation will depend upon the fermenting organism. EOF products include metabolites, such as citric acid, lactic acid, succinic acid, monosodium glutamate, gluconic acid, sodium gluconate, calcium gluconate, potassium gluconate, itaconic acid and other carboxylic acids, glucono delta-lactone, sodium erythorbate, lysine and other amino acids, omega 3 fatty acid, butanol, isoprene, 1,3-propanediol and other biomaterials.

### 5.6. Combination of Variants α-Amylases with Other Enzymes

Variant α-amylases may be combined with a glucoamylase (EC 3.2.1.3). Exemplary glucoamylases are from Trichoderma, Aspergillus, Talaromyces, Clostridium, Fusarium, Thielavia, Thermomyces, Athelia, Humicola, Penicillium, Artomyces, Gloeophyllum, Pycnoporus, Steccherinum, Trametes etc. Suitable commercial glucoamylases, include AMG 200L; AMG 300 L; SAN™ SUPER and AMG™ E (Novozymes); OPTIDEX® 300 and OPTIDEX L-400 (Danisco US Inc.); AMIGASE™ and AMIGASE™ PLUS (DSM); G-ZYME® G900 (Enzyme Bio-Systems); and G-ZYME® G990 ZR.

Other suitable enzymes that can be used with the variant α-amylase include phytase, protease, pullulanase, β-amylase, isoamylase, α-glucosidase, cellulase, xylanase, other hemicellulases, β-glucosidase, transferase, pectinase, lipase, cutinase, esterase, redox enzymes, a different α-amylase, or a combination thereof.

Compositions comprising the present α-amylases may be aqueous or non-aqueous formulations, granules, powders, gels, slurries, pastes, etc., which may further comprise any one or more of the additional enzymes listed, herein, along with buffers, salts, preservatives, water, co-solvents, surfactants, and the like.

### 6. Textile Desizing Compositions and Uses

Also contemplated are compositions and methods of treating fabrics (e.g., to desize a textile) using an amylase. Fabric-treating methods are well known in the art (see, e.g., U.S. Pat. No. 6,077,316). For example, the feel and appearance of a fabric can be improved by a method comprising contacting the fabric with an α-amylase in a solution. The fabric can be treated with the solution under pressure.

An α-amylase can be applied during or after the weaving of a textile, or during the desizing stage, or one or more additional fabric processing steps. During the weaving of textiles, the threads are exposed to considerable mechanical strain. Prior to weaving on mechanical looms, warp yarns are often coated with sizing starch or starch derivatives to increase their tensile strength and to prevent breaking. An α-amylase can be applied during or after the weaving to remove these sizing starch or starch derivatives. After weaving, an α-amylase can be used to remove the size coating before further processing the fabric to ensure a homogeneous and wash-proof result.

An α-amylase can be used alone or with other desizing chemical reagents and/or desizing enzymes to desize fabrics, including cotton-containing fabrics, as detergent additives, e.g., in aqueous compositions. An α-amylase also can be used in compositions and methods for producing a stonewashed look on indigo-dyed denim fabric and garments. For the manufacture of clothes, the fabric can be cut and sewn into clothes or garments, which are afterwards finished. In particular, for the manufacture of denim jeans, different enzymatic finishing methods have been developed. The finishing of denim garment normally is initiated with an enzymatic desizing step, during which garments are subjected to the action of amylolytic enzymes to provide softness to the fabric and make the cotton more accessible to the subsequent enzymatic finishing steps. An α-amylase can be used in methods of finishing denim garments (e.g., a “bio-stoning process”), enzymatic desizing and providing softness to fabrics, and/or finishing process.

### 7. Compositions and Methods for Baking and Food Preparation

The present compositions and method also relate to food composition, including but not limited to a food product, animal feed and/or food/feed additives, comprising the variant α-amylase, and methods for preparing such a food composition comprising mixing variant α-amylase with one or more food ingredients, or uses thereof. Furthermore, the present compositions and method relate to baking compositions, including but not limited to baker's flour, a dough, a baking additive and/or a baked product.

### 9. Brewing Compositions

The present variant α-amylase may be a component of a brewing composition used in a process of brewing, i.e., making a fermented malt beverage. Non-fermentable carbohydrates form the majority of the dissolved solids in the final beer. This residue remains because of the inability of malt amylases to hydrolyze the α-1,6-linkages of the starch. An α-amylase, optionally in combination with a glucoamylase and optionally a pullulanase and/or isoamylase, assists in converting the starch into dextrins and fermentable sugars, lowering the residual non-fermentable carbohydrates in the final beer.

All references cited herein are herein incorporated by reference in their entirety for all purposes. In order to further illustrate the compositions and methods, and advantages thereof, the following specific examples are given with the understanding that they are illustrative rather than limiting.

### EXAMPLE

### Example 1. AA2560 Variants

### Protein Expression, Purification and Quantitation:

AA2560 combinatorial variants in a ΔR181 and ΔG182 (i.e., ΔRG) background were made as synthetic genes and introduced into suitable Bacillus licheniformis cells using standard procedures. All mutations were confirmed by DNA sequencing. Cells were grown for 72 hours in a medium suitable for protein expression and secretion in a B. licheniformis host. Secreted protein was harvested by centrifugation. Purification was achieved through use of hydrophobic interaction chromatography with Phenyl Sepharose 6 Fast Flow resin (GE Healthcare). Purified proteins were stabilized in a standard formulation buffer containing HEPES as the buffering agent, calcium chloride, and propylene glycol at pH 8. Protein concentration was determined by a mixture of amino acid analysis, high performance liquid chromatography (HPLC) and absorbance at 280 nm.

### Enzyme Performance Assay:

The activity of the α-amylase was determined by removal of dyed starch stain from a white melamine tile in a detergent background. Mixed corn/rice colored starch tiles and mixed corn/rice starch tiles with food colorant, purchased from Center for Testmaterials (Catalog Nos. DM277 and DM71), respectively, were used to determine the cleaning activity of the α-amylase. The tiles were affixed to a 96-well plate containing the amylase solution diluted into a working range in an aqueous buffer and added to a pre-made detergent solution of the WFKB detergent (WFK Testgewebe GmbH, Brüggen, Deutschland) such that the total volume was 300 μL. Pre-imaged melamine tiles with colored starch stains were then affixed to the top of the 96 well plate, such that agitation of the assembly leads to splashing of the enzyme containing detergent onto the starch stained surface. The washing reaction was carried out at 50° C. for 15 minutes with shaking at 250 rpm. Following the washing reaction, the melamine tiles were then rinsed briefly under water, dried and re-imaged. The activity of the α-amylases is calculated as the difference in RGB (color) values of the pre and post wash images. The whiter the post wash image, the better the enzyme activity. Performance indices (PI) are calculated as:

$$\frac{change in RGB of variant}{change in RGB of wild type}$$

### Performance Indices of Combinatorial Variants Against the ΔRG Variant:

Cleaning performance of the variants in terms of performance index against the wild-type variant are listed in Table 3. DM277 is designated stain 1 and DM71 is designated stain 2.

TABLE 3
Variant performance on two different stains

| Mutations | PI-1 | PI-2 |
|---|---|---|
| ΔR181-ΔG182 | -1- | -1- |
| T40N-S91R-Q169H-ΔR181-ΔG182-T183M-H281N | 1.18 | 1.60 |
| Q172R-ΔR181-ΔG182-E190P-S288D | 1.09 | 1.36 |
| Q172R-ΔR181-ΔG182-S244E-S288D-S474R | 1.15 | 1.82 |
| S91R-Q172R-ΔR181-ΔG182-E190P-I324M | 1.26 | 1.95 |
| T40N-S91R-ΔR181-ΔG182-E190P-F263Y | 1.20 | 1.99 |
| T40N-S91R-ΔR181-ΔG182-S244E-Y364L | 1.20 | 2.19 |
| S91R-Q172R-ΔR181-ΔG182-E190P-I324R | 1.26 | 1.84 |
| S91R-W116R-Q172R-ΔR181-ΔG182-S244E-H281S -S288D | 1.29 | 2.19 |
| T40N-S91R-Y100F-W116R-Q172N-ΔR181-ΔG182-S244Q- | 1.24 | 1.88 |
| H281S | | |
| T40N-S91R-Q172R-ΔR181-ΔG182-S244Q-F263Y-H281S | 1.19 | 1.94 |
| S9IR-Q172R-ΔR181-ΔG182-E190P-I324N | 0.96 | 1.44 |
| T40D-S91R-Q172R-ΔRI 81-ΔG182-E190P-H281S-I324R | 1.04 | 0.99 |
| ΔR181-ΔG182-Y364L | 2.90 | 2.23 |

All variants in Table 2 perform equal to or better than STAINZYME® Plus 12L (Novozymes) in one or more of the stains and wash conditions tested.

### Example 2. Additioinal AA2560, AA707 and AAI10 Variants

To confirm the beneficial effects of the combinatorial mutations described in Example 1 in other parental molecules, additional AA2560, AA707, and AAI10 variants were made and tested as described. The molecules additionally included deletions at potions 181 and 182, 183 and 184, or did not include deletions. All mutations were confirmed by DNA sequencing.

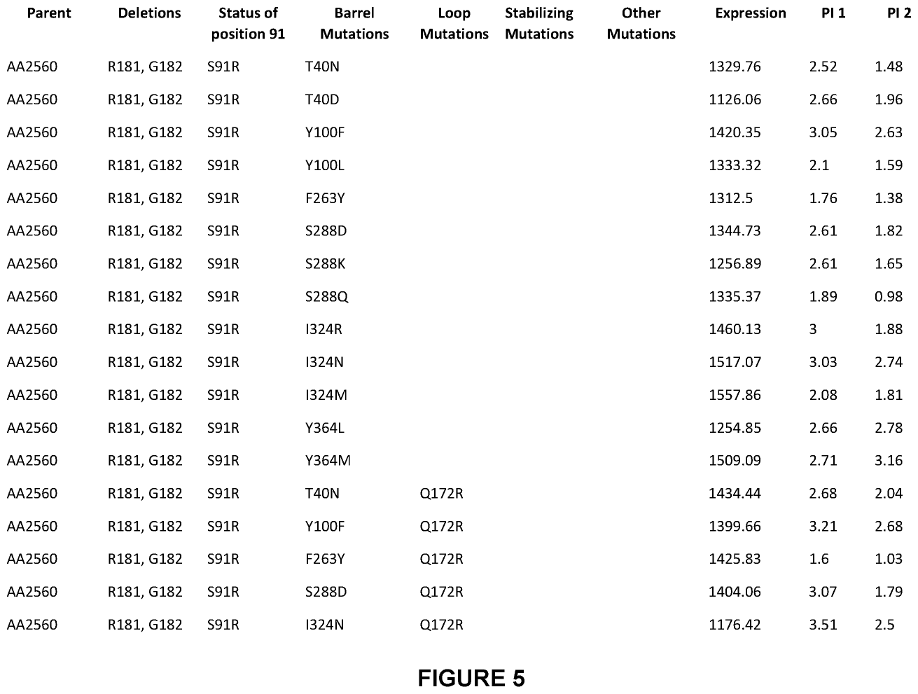

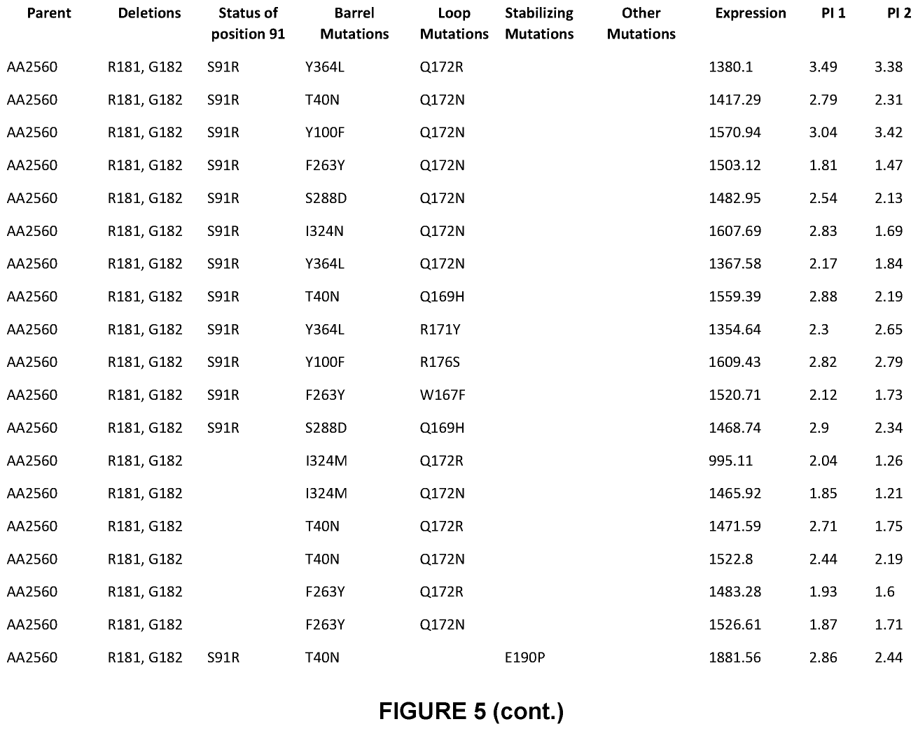

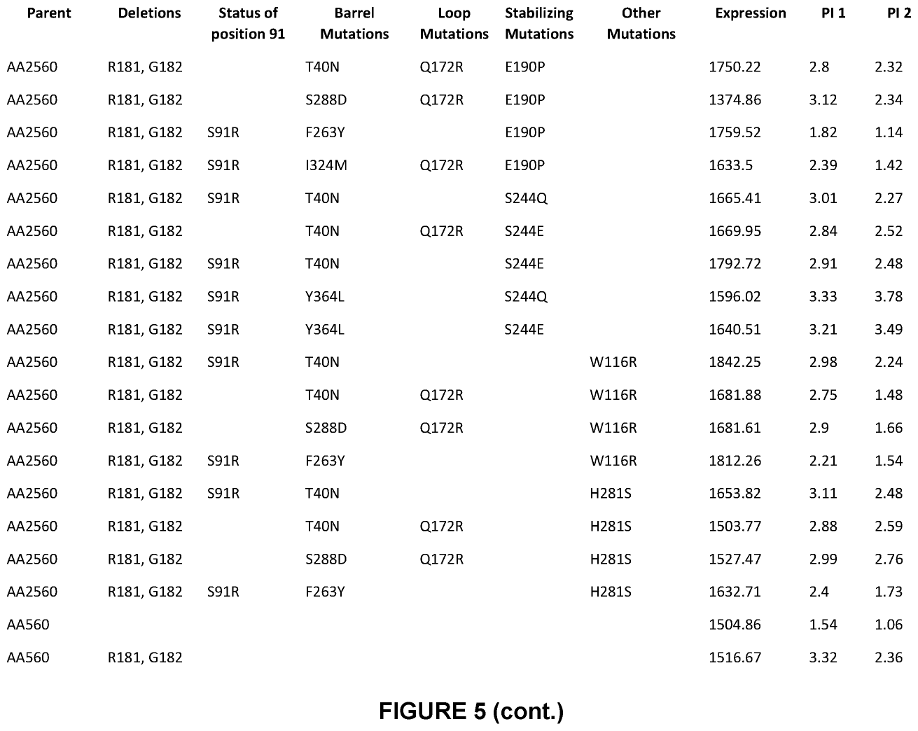

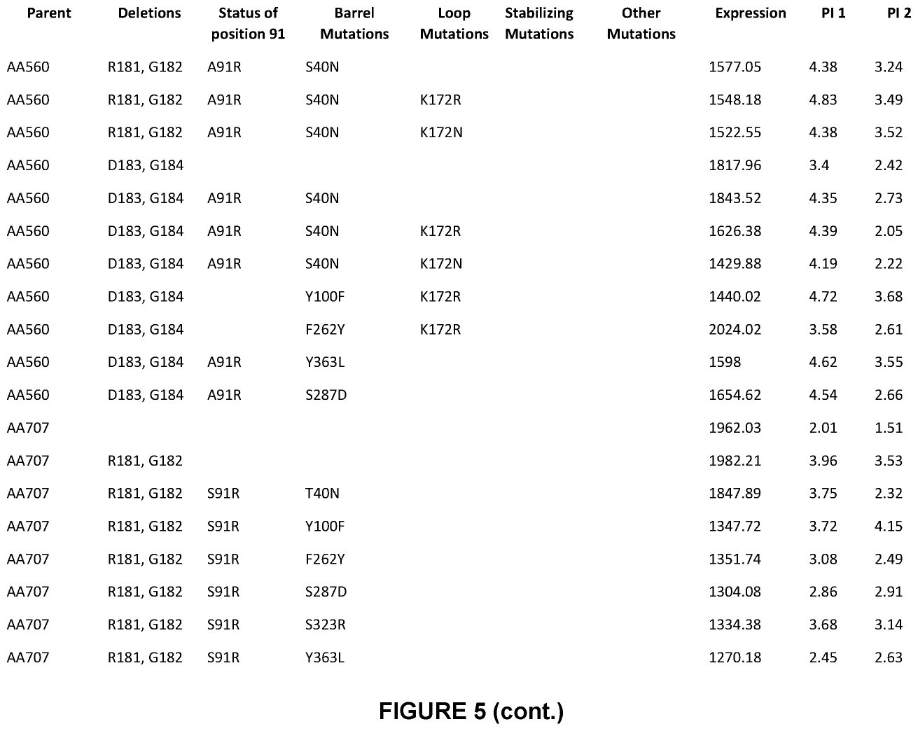

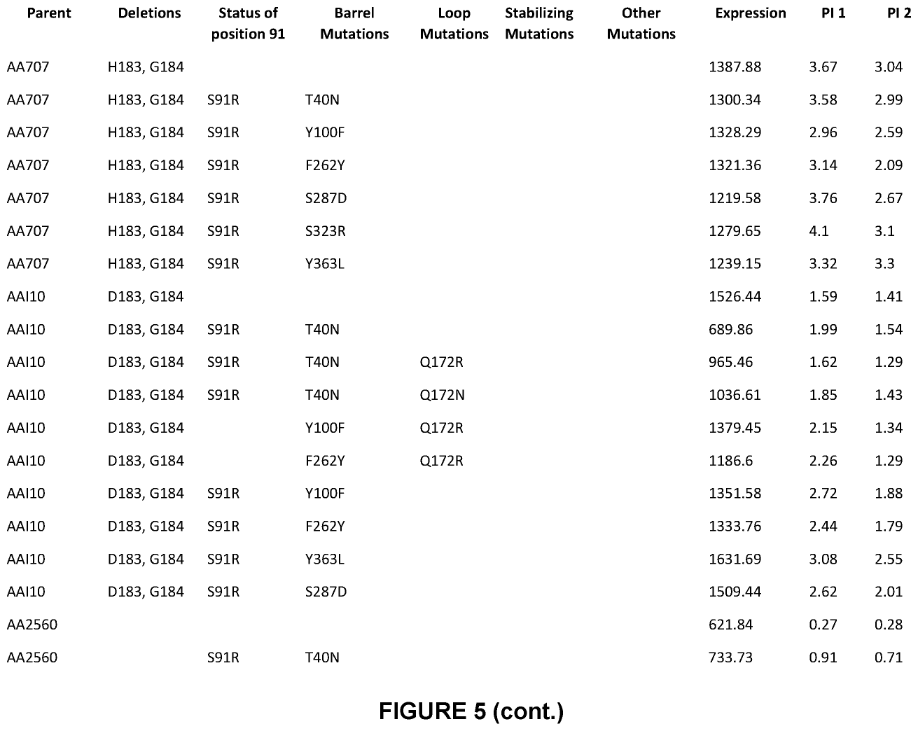

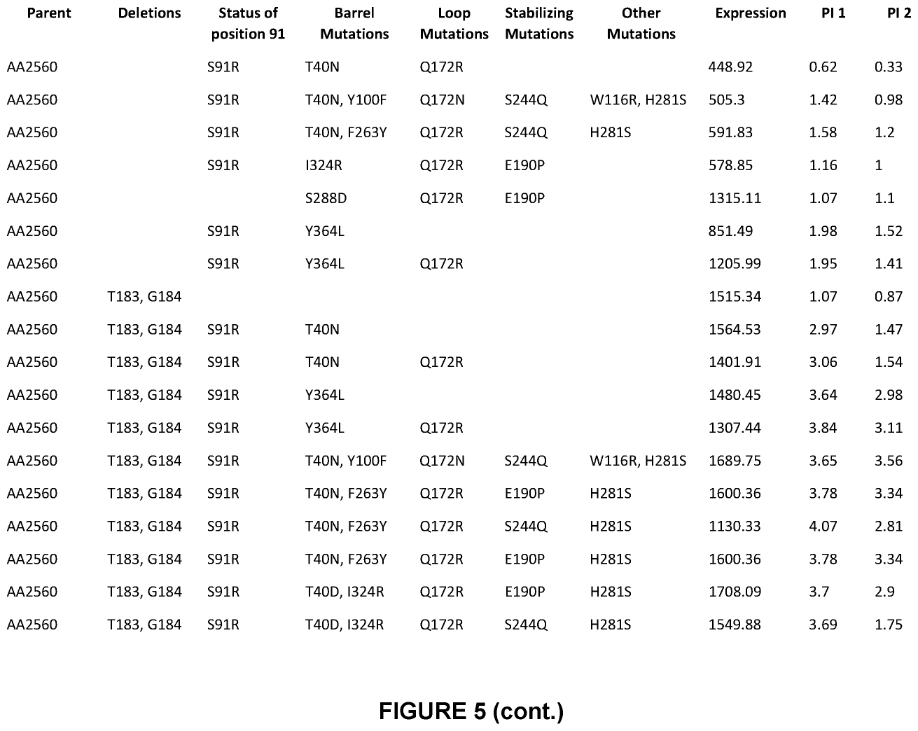

The performance of the variants was measured with respect to the closest parent, i.e., combinatorial variants in a “non-deletion” molecule were measured against the respective wild-type molecule. Combinatorial variants in a “double-delete” molecule were measured against the respective double-delete molecule. The results are shown in the Table in FIG. 5.

The results demonstrated a high degree of transferabilty of mutations across different parent molecules with the exception of AA707. An explanation for the different behavior of AA707 to mutations at the Ser91 position is proposed in the Detailed Description section of the document.

What is claimed is:

1. A recombinant, variant of a parent, non-naturally-occurring α-amylase molecule comprising a mutation at position 91 and a mutation at an amino acid residue at the base of the α-amylase TIM barrel structure, defined as residues 6, 7, 40, 96, 98, 100, 229, 230, 231, 262, 263, 285, 286, 287, 288, 322, 323, 324, 325, 362, 363 and 364, referring to SEQ ID NO: 1 for numbering, wherein the wild-type amino acid residue present at position 28 of the parent molecule is capable of taking on a positive charge.

2. The variant α-amylase of claim 1, wherein the mutation at position 91 is substitution of the naturally-present residue to a positively-charged residue.

3. The variant α-amylase of claim 1 or 2, wherein the mutation at position 91 is substitution of the naturally-present residue to arginine (i.e., X91R).

4. The variant α-amylase of any of claims 1-3, wherein the at least one mutation at the base of the α-amylase TIM barrel structure is selected from the group consisting of X40N, X40D, X100F, X100L, X263Y, X288D, X288K, X288Q, X324R, X324N, X324M, X364L and X364M.

5. The variant α-amylase of any of claims 1-4, wherein the at least one mutation at the base of the α-amylase TIM barrel structure is selected from the group consisting of T40N, T40D, Y100F, Y100L, F263Y, S288D, S288K, S288Q, I324R, I324N, I324M, Y364L and Y364M.

6. A recombinant, variant, non-naturally-occurring α-amylase comprising an arginine at position 91 and at least one of the following features not present in naturally-occurring α-amylase: N or D at position 40, F or L at position 100, Y at position 263, D, K or Q at position 288, R, N or M at position 324 or L or M at position 364.

7. The variant α-amylase of any of claims 1-6, further comprising a mutation at a residue in the loop comprising surface-exposed residues 167, 169, 171, 172 and 176, referring to SEQ ID NO: 1 for numbering.

8. The variant α-amylase of claim 7, wherein the at least one mutation in the loop is selected from the group consisting of X167F, X169H, X171Y, X172R, X172N and X176S.

9. The variant α-amylase of claim 8, wherein the at least one mutation in the loop is selected from the group consisting of W167F, Q169H, R171Y, Q172R, Q172N and R176S.

10. The variant α-amylase of any of claims 1-6, further comprising F at position 167, H at position 169, Y at position 171, R or N at position 172 or S at position 176, referring to SEQ ID NO: 1 for numbering.

11. A recombinant, variant, non-naturally-occurring α-amylase comprising a mutation at position 172 and a mutation at position 288, referring to SEQ ID NO: 1 for numbering.

12. A recombinant, variant, non-naturally-occurring α-amylase comprising arginine or asparagine at position 172 and aspartic acid at position 288, referring to SEQ ID NO: 1 for numbering.

13. The variant α-amylase of any of claims 1-12, further comprising a mutation at position 116 and/or 281, referring to SEQ ID NO: 1 for numbering.

14. The variant α-amylase of any of claims 1-12, further comprising arginine at position 116 or serine at position 281, referring to SEQ ID NO: 1 for numbering.

15. The variant α-amylase of any of any of claims 1-14, further comprising a mutation at position 190 and/or 244, referring to SEQ ID NO: 1 for numbering.

16. The variant α-amylase of any of any of claims 1-14, having proline at position 190 and/or alanine, glutamic acid or glutamine at position 244, referring to SEQ ID NO: 1 for numbering.

17. The variant α-amylase of any of claims 1-16, further comprising deletion of at least two residues equivalent to R181, G182, T183, and G184, using SEQ ID NO: 1.

18. The variant α-amylase of any of claims 1-16, further comprising pairwaise deletions of residues equivalent to R181 and G182 or to residues T183 and G184.

19. A recombinant, variant, non-naturally-occurring α-amylase comprising:
(i) substitutions selected from the group consisting of:
(a) X40N-X91R-X169H-X183M-X281N,
(b) X172R-X190P-X288D,
(c) X172R-X244E-X288D-X474R,
(d) X91R-X172R-X190P-X324M,
(e) X40N-X91R-X190P-X263Y,
(f) X40N-X91R-X244E-X364L,
(g) X91R-X172R-X190P-X324R,
(h) X91R-X116R-X172R-X244E-X281S-X288D,
(i) X40N-X91R-X100E-X116R-X172N-X244Q-X281S,
(j) X40N-X91R-X172R-X244Q-X263Y-X281S,
(k) X91R-X172R-X190P-X324N,
(l) X40D-X91R-X172R-X190P-X281S-X324R, and
(m) X364L; and
(ii) pairwaise deletions of residues selected from the group consisting of residues equavalent to:
181 and 182, and
183 and 184,
using SEQ ID NO: 1 for numbering.

20. The recombinant, variant, non-naturally-occurring α-amylase of claim 19 comprising:
(i) substitutions selected from the group consisting of:
(a) T40N-S91R-Q169H-T183M-H281N,
(b) Q172R-E190P-S288D,
(c) Q172R-S244E-S288D-S474R,
(d) S91R-Q172R-E190P-I324M,
(e) T40N-S91R-E190P-F263Y,
(f) T40N-S91R-S244E-Y364L,
(g) S91R-Q172R-E190P-I324R,
(h) S91R-W116R-Q172R-S244E-H281S-S288D,
(i) T40N-S91R-Y100E-W116R-Q172N-S244Q-H281S,
(j) T40N-S91R-Q172R-S244Q-F263Y-H281S,
(k) S91R-Q172R-E190P-I324N,
(1) T40D-S91R-Q172R-E190P-H281S-I324R, and
(m) Y364L; and
(ii) pairwaise deletions of residues selected from the group consisting of:
R181 and G182, and
T183 and G184,
using SEQ ID NO: 1 for numbering.

21. A recombinant, variant, non-naturally-occurring α-amylase comprising three or more of the following features: (a) D or N at position 40 and/or R at position 91 and (b) F at position 100, Y at position 263, D at position 288, M, N or R at position 324 and/or L at position 364, optionally in combination with (c) H at position 169, M at position 183M, N or S at position 281, N or R at position 172, P at position 190, E, Q or R at position 244, R at position 474 and/or R at postion 116, (d) optionally in combination with (e) pairwise deletions at positions 181 and 182 or 183 and 184, in all cases using SEQ ID NO: 1 for numbering.

22. The variant α-amylase of any of claims 1-21, having at least 70%, at least 80%, at least 90% or at least 95% amino acid sequence identity to the amino acid sequence of SEQ ID NO: 1, SEQ ID NO: 2, SEQ ID NO: 3 or SEQ ID NO: 4.

23. A detergent composition comprising the variant α-amylase of any of claims 1-22.

24. The detergent composition of claim 23, further comprising a variant subtilisin protease from Bacillus gibsonii having the amino acid substitutions X39E, X99R, X126A, X127E and X128G, and further comprising one or more additional substitutions selected from the group consisting of N74D-M211L-N253P, R179Q-M211L-N253P, N74D-N253P, N85R-G160Q-R179Q-M211L-N212S-N253P, R179Q-N253P, G160Q-R179Q-M211L-N212S-N253P, R179Q-M211L, G160Q-R179Q-M211L-N253P, G160Q-R179Q-N212S-N253P, N74D-M211L, M211L-N242D, G160Q-R179Q-M211L-N212S, N74D-R179Q-M211L-N253P, G160Q-R179Q-M211L, G160Q-R179Q-N253P, N74D-Q200L-M211L, N74D-G160Q-N212S-N253P, N74D-G160Q-M211L-N253P, G160Q-R179Q, G160Q-R179Q-N212S, N74D-G160Q-N253P, N74D-G160Q-R179Q-M211L-N212S-N253P, N74D-N085R-G160Q-R179Q-M211L, N74D-G160Q-M211L-N212S-N253P, N74D-N085R-N116R-Q200L-Q256E, N74D-G160Q-R179Q-N212S-N253P, N74D-G160Q-M211L-N212S, N74D-G160Q, N74D-G160Q-R179Q-M211L-N253P, N74D-R179Q-M211L, N74D-G160Q-N212S, N74D-G160Q-M211L, N74D-G160Q-R179Q-N253P, N74D, N74D-G160Q-R179Q-M211L-N212S, N74D-N085R-M211L-N212S, N74D-G160Q-R179Q-N212S, N74D-G160Q-R179Q-M211L, N74D-M211L-Q256E, N74D-G160Q-R179Q, R179Q-M211L-N212S-N253P, R179Q-M211L-N212S, N74D-N085R-R179Q-M211L-N212S, N74D-M211L-N212S, N74D-R179Q-M211L-N212S, N74D-M211L-N242D, N74D-Q200L-M211L-Q256E, N74D-Q200L-M211L-N242D-Q256E, N74D-Q200L, N74D-M211N-N212Q, N74D-M211N-N212Q-Q256E, N74D-M211N-Q256E, N74D-M211Q, N74D-M211Q-N212Q, N74D-M211Q-N212Q-Q256E, N74D-M211Q-Q256E, N74D-N198A-M211Q, N74D-N198A-M211Q-N212Q, N74D-N198A-M211Q-Q256E, N74D-N198G-M211Q, N74D-N198G-M211Q-N212Q, N74D-N198G-M211Q-Q256E, N74D-N198K-M211Q-N212Q, N74D-N198L-M211Q-N212Q, N74D-N198Q-M211Q-N212Q, N74D-N198R-M211Q-N212Q, N74D-N198T-M211Q-N212Q, N74D-N198V-M211Q-N212Q, N74D-N212Q-Q256E, N74D-Q256E, N74D-R207Q, N74D-R207Q-M211N, N74D-R207Q-M211N-N212Q, N74D-R207Q-M211N-N212Q-Q256E, N74D-R207Q-M211N-Q256E, N74D-R207Q-M211Q, N74D-R207Q-M211Q-N212Q, N74D-R207Q-M211Q-N212Q-Q256E, N74D-R207Q-N212Q, N74D-R207Q-N212Q-Q256E, N74D-R207Q-Q256E, N74D-N198S-M211Q and N74D-N198L-M211Q, referring to SEQ ID NO: 5 for numbering and having at least 90% amino acid sequence identity to SEQ ID NO: 6.

25. A method for converting starch to oligosaccharides, comprising contacting starch with effective amount of the variant α-amylase of any of claims 1-22.

26. A method for removing a starchy stain or soil from a surface, comprising contacting the surface with an effective amount of the variant α-amylase of any of claims 1-22, and allowing the polypeptide to hydrolyze starch components present in the starchy stain to produce smaller starch-derived molecules that dissolve in the aqueous composition, thereby removing the starchy stain from the surface.

27. A nucleic acid encoding the variant α-amylase of any of claims 1-22.

28. A host cell comprising the nucleic acid of claim 27.
